SEQUENCE LISTING (12 sequences)

SEQ ID NO: 1 - DNA, 380 bp, Artificial Sequence
  CMV enhancer
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 2 - DNA, 203 bp, Artificial Sequence
  CMV promoter
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgc

SEQ ID NO: 3 - DNA, 181 bp, Artificial Sequence
  Truncated 5′ LTR
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 4 - DNA, 256 bp, Artificial Sequence
  EFS promoter
taggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagg

SEQ ID NO: 5 - DNA, 66 bp, Artificial Sequence
  P2A linker sequence
ggatccggcgcaacaaacttctctctgctgaaacaagccggagatgtcgaagagaatcctggaccg

SEQ ID NO: 6 - DNA, 399 bp, Artificial Sequence
  Blasticidin resistance gene
atggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaa

SEQ ID NO: 7 - DNA, 503 bp, Artificial Sequence
  3′ LTR sequence with a partially deleted U3 region
ctggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcag

SEQ ID NO: 8 - DNA, 3630 bp, Artificial Sequence
  EGFR sequence
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagca

SEQ ID NO: 9 - DNA, 4152 bp, Artificial Sequence
  Cas9 sequence
atggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctactggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaa

SEQ ID NO: 10 - DNA, 1122 bp, Artificial Sequence
  CCR2 sequence
atgctgtccacatctcgttctcggtttatcagaaataccaacgagagcggtgaagaagtcaccaccttttttgattatgattacggtgctccctgtcataaatttgacgtgaagcaaattggggcccaactcctgcctccgctctactcgctggtgttcatctttggttttgtgggcaacatgctggtcgtcctcatcttaataaactgcaaaaagctgaagtgcttgactgacatttacctgctcaacctggccatctctgatctgctttttcttattactctcccattgtgggctcactctgctgcaaatgagtgggtctttgggaatgcaatgtgcaaattattcacagggctgtatcacatcggttattttggcggaatcttcttcatcatcctcctgacaatcgatagatacctggctattgtccatgctgtgtttgctttaaaagccaggacggtcacctttggggtggtgacaagtgtgatcacctggttggtggctgtgtttgcttctgtcccaggaatcatctttactaaatgccagaaagaagattctgtttatgtctgtggcccttattttccacgaggatggaataatttccacacaataatgaggaacattttggggctggtcctgccgctgctcatcatggtcatctgctactcgggaatcctgaaaaccctgcttcggtgtcgaaacgagaagaagaggcatagggcagtgagagtcatcttcaccatcatgattgtttactttctcttctggactccctataatattgtcattctcctgaacaccttccaggaattcttcggcctgagtaactgtgaaagcaccagtcaactggaccaagccacgcaggtgacagagactcttgggatgactcactgctgcatcaatcccatcatctatgccttcgttggggagaagttcagaagcctttttcacatagctcttggctgtaggattgccccactccaaaaaccagtgtgtggaggtccaggagtgagaccaggaaagaatgtgaaagtgactacacaaggactcctcgatggtcgtggaaaaggaaagtcaattggcagagcccctgaagccagtcttcaggacaaagaaggagcc

SEQ ID NO: 11 - DNA, 72 bp, Artificial Sequence
  Flag sequence
gattacaaagacgatgacgataaggattacaaagacgatgacgataaggattacaaagacgatgacgataag

SEQ ID NO: 12 - DNA, 723 bp, Artificial Sequence
  EGFP sequence
gccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

(19) United States
(12) Patent Application Publication
(10) Pub. No.: US 2023/0212607 A1
(43) Pub. Date: Jul. 6, 2023

(54) IMPROVED LENTIVIRAL EXPRESSION VECTOR, CONSTRUCTION METHOD FOR SAME, AND APPLICATIONS THEREOF

(71) Applicant: NANJING GENSCRIPT BIOTECH CO., LTD., Nanjing, Jiangsu (CN)

(72) Inventors: Jiangbo Zou, Jiangsu (CN); Haiyu Fang, Jiangsu (CN)

(21) Appl. No.: 17/998,553

(22) PCT Filed: May 13, 2021
(86) PCT No.: PCT/CN2021/093495

§ 371 Date: Nov. 11, 2022

(30) Foreign Application Priority Data
May 13, 2020 (CN) 202010402053.1

(51) Int. Cl. C12N 15/86

(52) U.S. Cl. CPC C12N 15/86; C12N 2740/15041; C12N 2740/00041

(57) ABSTRACT

Provided are an improved lentiviral expression vector, a construction method for same, and applications thereof. The plasmid backbone of the lentiviral expression vector is based on pLVX-Puro and is improved via the following steps: a. adding a CMV enhancer and promoter to the upstream of 5′ LTR by means of gene synthesis and subcloning; b. replacing the original 5′ LTR with a new 5′ LTR (SEQ ID NO: 3) of which the length is 181 bps; c. replacing the CMV promoter on an original vector with an EFS promoter; d. deleting a PGK promoter after multiple cloning sites of the original vector and, at the same time, introducing a P2A connection subsequence; e. replacing original resistance against Puro with resistance against a shorter Blasticidin; and f. replacing original 3′ LTR with an LTR (SEQ ID NO: 7) with a partially deleted U3. The lentiviral expression vector acquired by the improvement provides a greater packaging capacity in comparison with pLVX-Puro.

### TECHNICAL FIELD

The present invention belongs to the field of molecular biology, and in particular relates to an improved lentiviral expression vector, a construction method for same, and applications thereof.

### BACKGROUND ART

Lentiviruses are a genus of retroviruses, and the most common one is derived from HIV viruses. Lentiviruses are chemically single-stranded RNA viruses with a genome length of approximately 9 kb and contain three major elements, namely Gag expressing a coat protein, Pol expressing a protease, an integrase and a reverse transcriptase, and Rev expressing an envelope protein. Among them, the Gag protein first expresses three main structural proteins, namely a matrix protein, a coat protein and a nucleocapsid protein by means of alternative splicing, and then assists the virus replication by means of an enzyme system expressed by the Pol protein, and finally forms a mature virus with the participation of the envelope protein. The envelope protein on the surface of the virus binds directly to a receptor and then enters a cell by means of membrane fusion. Lentiviruses are broadly invasive and can infect most mammalian cells. After the cells are infected, cDNA can be obtained by reverse transcription, and then the genome is randomly integrated into a host genome to form a stably expressed cell line. At the same time, unlike gammaretroviruses which can only infect dividing cells, lentiviruses can infect both dividing and non-dividing cells, so lentiviruses have become a particularly important tool for gene therapy and cell therapy.

In order to continuously increase the safety of lentiviral vectors, lentiviral vectors have gone through three generations since their discovery. At present, the second-generation lentiviral system and the third-generation lentiviral system are more commonly used. The second-generation lentiviral system is a three-plasmid system, including an expression plasmid expressing a target gene, a packaging plasmid expressing Tat-rev, and an envelope plasmid expressing an envelope protein. The third-generation lentiviral system is further improved on the basis of the second-generation lentiviral system, and is no longer dependent on a Tat protein by deleting a U3 region at the 5′ LTR end and fusing a promoter upstream. In addition. the ability of the virus to replicate itself is reduced by partial deletion of U3 at the 3′ LTR end, while the risk of forming a wild-type virus is further reduced by expressing Gag-pol and Rev in two plasmids, respectively.

Due to the limitation of the lentiviral genome, at present, the upper limit of the length of the recombinant lentiviral expression vector is approximately in a range of 9-10 kb. If some necessary regulatory elements and related promoters, terminators, etc. are removed, the carrying capacity of the lentiviral expression vector is generally about 3 kb, and if the carrying capacity exceeds 3 kb, a high titer is difficult to achieve (see: Plasmids 101: A Desktop Resource. Created and Compiled by Addgene, October 2015, 2nd Edition). However, the length of many important genes such as EGFR exceeds the upper limit of the packaging capacity of a conventional lentivirus, which seriously limits our research on the function of such genes.

### SUMMARY OF THE INVENTION

In order to solve the problem of small packaging capacity (within 3 kb) of traditional lentiviral expression vectors, the present invention develops an improved lentiviral expression vector. By means of improvement and optimization of the PLVX-Puro lentiviral expression vector, for example, adjustment, deletion and modification of some elements thereof, the carrying capacity of the lentiviral expression vector is greatly increased. which allows for packaging genes longer than 5 kb and obtaining a higher titer. This improved lentiviral expression vector is suitable for the third-generation lentiviral packaging system, which will provide a more convenient tool for the research on long-fragment gene functions.

In one aspect, the present invention provides a lentiviral expression vector improved on the basis of PLVX-Puro, which is characterized in that the vector comprises at least one, at least two or three of the following improved element sequences:

(1) a CMV promoter sequence upstream of 5′ LTR,
(2) an EFS promoter sequence as a target gene promoter, and
(3) a P2A linker sequence downstream of a multiple cloning site.

In some embodiments, the vector comprises the improved element sequences: (1) an EFS promoter sequence as a target gene promoter, and (2) a P2A linker sequence downstream of a multiple cloning site. In other embodiments, the vector comprises the improved element sequences: (1) a CMV promoter sequence upstream of 5′ LTR. (2) an EFS promoter sequence as a target gene promoter, and (3) a P2A linker sequence downstream of a multiple cloning site.

Further, the vector comprises a CMV enhancer sequence upstream of the CMV promoter sequence upstream of the 5′ LTR.

In some embodiments, the 5′ LTR is a truncated PLVX-Puro 5′ LTR sequence.

In some embodiments, the lentiviral expression vector further comprises a 3′ LTR sequence with a partially deleted U3 region.

In some embodiments, the CMV promoter sequence upstream of the 5′ LTR comprises a nucleotide sequence shown in SEQ ID NO: 2 or a nucleotide sequence that has at least 80% identity to the nucleotide sequence shown in SEQ ID NO: 2 and has a promoter activity. In other embodiments, the CMV promoter sequence upstream of the 5′ LTR comprises a nucleotide sequence that has at least 80%, at least 83%, at least 85%, at least 87%, at least 89%. at least 91%, at least 93%, at least 95%, at least 97% or at least 99% identity to the nucleotide sequence shown in SEQ ID NO: 2 and has a promoter activity. In some embodiments, the CMV promoter sequence upstream of the 5′ LTR comprises a nucleotide sequence that has at least 80% identity to the nucleotide sequence shown in SEQ ID NO: 2 and has a promoter activity. In other embodiments, the CMV promoter sequence upstream of the 5′ LTR comprises a nucleotide sequence that has at least 85% identity to the nucleotide sequence shown in SEQ ID NO: 2 and has a promoter activity. In some embodiments. the CMV promoter sequence upstream of the 5′ LTR comprises a nucleotide sequence that has at least 90% identity to the nucleotide sequence shown in SEQ ID NO: 2 and has a promoter activity. In other embodiments, the CMV promoter sequence upstream of the 5′ LTR comprises a nucleotide sequence that has at least 95% identity to the nucleotide sequence shown in SEQ ID NO: 2 and has a promoter activity. In some embodiments, the CMV promoter sequence upstream of the 5′ LTR comprises a nucleotide sequence that has at least 97% identity to the nucleotide sequence shown in SEQ ID NO: 2 and has a promoter activity. In other embodiments, the CMV promoter sequence upstream of the 5′ LTR comprises a nucleotide sequence that has at least 99% identity to the nucleotide sequence shown in SEQ ID NO: 2 and has a promoter activity. In a specific embodiment, the CMV promoter sequence upstream of the 5′ LTR comprises the nucleotide sequence shown in SEQ ID NO: 2. In another specific embodiment, the CMV promoter sequence upstream of the 5′ LTR is the nucleotide sequence shown in SEQ ID NO: 2.

In some embodiments, the EFS promoter sequence comprises a nucleotide sequence shown in SEQ ID NO: 4 or a nucleotide sequence that has at least 80% identity to the nucleotide sequence shown in SEQ ID NO: 4 and has a promoter activity. In other embodiments, the EFS promoter sequence comprises a nucleotide sequence that has at least 80%, at least 83/o, at least 85%, at least 87%, at least 89%, at least 91%, at least 93%, at least 95%, at least 97% or at least 99% identity to the nucleotide sequence shown in SEQ ID NO: 4 and has a promoter activity. In some embodiments, the EFS promoter sequence comprises a nucleotide sequence that has at least 80% identity to the nucleotide sequence shown in SEQ ID NO: 4 and has a promoter activity. In other embodiments, the EFS promoter sequence comprises a nucleotide sequence that has at least 83% identity to the nucleotide sequence shown in SEQ ID NO: 4 and has a promoter activity. In some embodiments. the EFS promoter sequence comprises a nucleotide sequence that has at least 85% identity to the nucleotide sequence shown in SEQ ID NO: 4 and has a promoter activity. In other embodiments, the EFS promoter sequence comprises a nucleotide sequence that has at least 87% identity to the nucleotide sequence shown in SEQ ID NO: 4 and has a promoter activity. In some embodiments, the EFS promoter sequence comprises a nucleotide sequence that has at least 89% identity to the nucleotide sequence shown in SEQ ID NO: 4 and has a promoter activity. In other embodiments, the EFS promoter sequence comprises a nucleotide sequence that has at least 91% identity to the nucleotide sequence shown in SEQ ID NO: 4 and has a promoter activity. In other embodiments, the EFS promoter sequence comprises a nucleotide sequence that has at least 93% identity to the nucleotide sequence shown in SEQ ID NO: 4 and has a promoter activity. In some embodiments, the EFS promoter sequence comprises a nucleotide sequence that has at least 95% identity to the nucleotide sequence shown in SEQ ID NO: 4 and has a promoter activity. In other embodiments, the EFS promoter sequence comprises a nucleotide sequence that has at least 97% identity to the nucleotide sequence shown in SEQ ID NO: 4 and has a promoter activity. In some embodiments, the EFS promoter sequence comprises a nucleotide sequence that has at least 99% identity to the nucleotide sequence shown in SEQ ID NO: 4 and has a promoter activity. In a specific embodiment, the EFS promoter sequence comprises the nucleotide sequence shown in SEQ ID NO: 4. In another specific embodiment, the EFS promoter sequence is the nucleotide sequence shown in SEQ ID NO: 4.

In some embodiments, the P2A linker sequence comprises a nucleotide sequence shown in SEQ ID NO: 5 or a nucleotide sequence that has at least 80% identity to the nucleotide sequence shown in SEQ ID NO: 5 and has a ligation function. In other embodiments, the P2A linker sequence comprises a nucleotide sequence that has at least 80%, at least 83/o, at least 85%, at least 87%, at least 89%, at least 91%, at least 93%, at least 95%, at least 97% or at least 99% identity to the nucleotide sequence shown in SEQ ID NO: 5 and has a ligation function. In some embodiments, the P2A linker sequence comprises a nucleotide sequence that has at least 80% identity to the nucleotide sequence shown in SEQ ID NO: 5 and has a ligation function. In other embodiments. the P2A linker sequence comprises a nucleotide sequence that has at least 83% identity to the nucleotide sequence shown in SEQ ID NO: 5 and has a ligation function. In some embodiments, the P2A linker sequence comprises a nucleotide sequence that has at least 85% identity to the nucleotide sequence shown in SEQ ID NO: 5 and has a ligation function. In other embodiments, the P2A linker sequence comprises a nucleotide sequence that has at least 87% identity to the nucleotide sequence shown in SEQ ID NO: 5 and has a ligation function. In some embodiments, the P2A linker sequence comprises a nucleotide sequence that has at least 89% identity to the nucleotide sequence shown in SEQ ID NO: 5 and has a ligation function. In other embodiments, the P2A linker sequence comprises a nucleotide sequence that has at least 91% identity to the nucleotide sequence shown in SEQ ID NO: 5 and has a ligation function. In some embodiments, the P2A linker sequence comprises a nucleotide sequence that has at least 93% identity to the nucleotide sequence shown in SEQ ID NO: 5 and has a ligation function. In other embodiments, the P2A linker sequence comprises a nucleotide sequence that has at least 95% identity to the nucleotide sequence shown in SEQ ID NO: 5 and has a ligation function. In some embodiments, the P2A linker sequence comprises a nucleotide sequence that has at least 97% identity to the nucleotide sequence shown in SEQ ID NO: 5 and has a ligation function. In other embodiments, the P2A linker sequence comprises a nucleotide sequence that has at least 99% identity to the nucleotide sequence shown in SEQ ID NO: 5 and has a ligation function. In a specific embodiment, the P2A linker sequence comprises the nucleotide sequence shown in SEQ ID NO: 5. In another specific embodiment, the P2A linker sequence is the nucleotide sequence shown in SEQ ID NO: 5.

In some embodiments, the CMV enhancer sequence comprises a nucleotide sequence shown in SEQ ID NO: 1 or a nucleotide sequence that has at least 80% identity to the nucleotide sequence shown in SEQ ID NO: 1 and has an enhancer activity. In other embodiments, the CMV enhancer sequence comprises a nucleotide sequence that has at least 80%, at least 83/a, at least 85%, at least 87%, at least 89%, at least 91%, at least 93%, at least 95%, at least 97% or at least 99% identity to the nucleotide sequence shown in SEQ ID NO: 1 and has an enhancer activity. In some embodiments, the CMV enhancer sequence comprises a nucleotide sequence that has at least 80% identity to the nucleotide sequence shown in SEQ ID NO: 1 and has an enhancer activity. In other embodiments, the CMV enhancer sequence comprises a nucleotide sequence that has at least 83% identity to the nucleotide sequence shown in SEQ ID NO: 1 and has an enhancer activity. In some embodiments. the CMV enhancer sequence comprises a nucleotide sequence that has at least 85% identity to the nucleotide sequence shown in SEQ ID NO: 1 and has an enhancer activity. In other embodiments, the CMV enhancer sequence comprises a nucleotide sequence that has at least 87% identity to the nucleotide sequence shown in SEQ ID NO: 1 and has an enhancer activity. In some embodiments, the CMV enhancer sequence comprises a nucleotide sequence that has at least 89% identity to the nucleotide sequence shown in SEQ ID NO: 1 and has an enhancer activity. In some embodiments, the CMV enhancer sequence comprises a nucleotide sequence that has at least 91% identity to the nucleotide sequence shown in SEQ ID NO: 1 and has an enhancer activity. In other embodiments, the CMV enhancer sequence comprises a nucleotide sequence that has at least 93% identity to the nucleotide sequence shown in SEQ ID NO: 1 and has an enhancer activity. In some embodiments, the CMV enhancer sequence comprises a nucleotide sequence that has at least 95% identity to the nucleotide sequence shown in SEQ ID NO: 1 and has an enhancer activity. In other embodiments, the CMV enhancer sequence comprises a nucleotide sequence that has at least 97% identity to the nucleotide sequence shown in SEQ ID NO: 1 and has an enhancer activity. In some embodiments, the CMV enhancer sequence comprises a nucleotide sequence that has at least 99% identity to the nucleotide sequence shown in SEQ ID NO: 1 and has an enhancer activity. In a specific embodiment, the CMV enhancer sequence comprises the nucleotide sequence shown in SEQ ID NO: 1. In another specific embodiment, the CMV enhancer sequence is the nucleotide sequence shown in SEQ ID NO: 1.

In some embodiments, the truncated PLVX-Puro 5′ LTR sequence comprises a nucleotide sequence shown in SEQ ID NO: 3 or a nucleotide sequence that has at least 80% identity to the nucleotide sequence shown in SEQ ID NO: 3 and can function as the 5′ LTR. In other embodiments, the truncated 5′ LTR sequence comprises a nucleotide sequence that has at least 80%, at least 83%, at least 85%, at least 87%, at least 89%, at least 91%, at least 93%, at least 95%, at least 97% or at least 99% identity to the nucleotide sequence shown in SEQ ID NO: 3 and can function as the 5′ LTR. In some embodiments, the truncated 5′ LTR sequence comprises a nucleotide sequence that has at least 80% identity to the nucleotide sequence shown in SEQ ID NO: 3 and can function as the 5′ LTR. In other embodiments, the truncated 5′ LTR sequence comprises a nucleotide sequence that has at least 83% identity to the nucleotide sequence shown in SEQ ID NO: 3 and can function as the 5′ LTR. In some embodiments, the truncated 5′ LTR sequence comprises a nucleotide sequence that has at least 85% identity to the nucleotide sequence shown in SEQ ID NO: 3 and can function as the 5′ LTR. In other embodiments, the truncated 5′ LTR sequence comprises a nucleotide sequence that has at least 87% identity to the nucleotide sequence shown in SEQ ID NO: 3 and can function as the 5′ LTR. In some embodiments, the truncated 5′ LTR sequence comprises a nucleotide sequence that has at least 89% identity to the nucleotide sequence shown in SEQ ID NO: 3 and can function as the 5′ LTR. In other embodiments, the truncated 5′ LTR sequence comprises a nucleotide sequence that has at least 91% identity to the nucleotide sequence shown in SEQ ID NO: 3 and can function as the 5′ LTR. In some embodiments, the truncated 5′ LTR sequence comprises a nucleotide sequence that has at least 93% identity to the nucleotide sequence shown in SEQ ID NO: 3 and can function as the 5′ LTR. In other embodiments, the truncated 5′ LTR sequence comprises a nucleotide sequence that has at least 95% identity to the nucleotide sequence shown in SEQ ID NO: 3 and can function as the 5′ LTR. In some embodiments, the truncated 5′ LTR sequence comprises a nucleotide sequence that has at least 97% identity to the nucleotide sequence shown in SEQ ID NO: 3 and can function as the 5′ LTR. In other embodiments, the truncated 5′ LTR sequence comprises a nucleotide sequence that has at least 99% identity to the nucleotide sequence shown in SEQ ID NO: 3 and can function as the 5′ LTR. In a specific embodiment, the truncated 5′ LTR sequence comprises the nucleotide sequence shown in SEQ ID NO: 3. In another specific embodiment, the truncated 5′ LTR sequence is the nucleotide sequence shown in SEQ ID NO: 3.

In some embodiments, the 3′ LTR sequence with the partially deleted U3 region of the present invention comprises a nucleotide sequence shown in SEQ ID NO: 7 or a nucleotide sequence that has at least 80% identity to the nucleotide sequence shown in SEQ ID NO: 7 and can function as the 3′ LTR. In other embodiments, the 3′ LTR sequence with the partially deleted U3 region comprises a nucleotide sequence that has at least 80%, at least 83%, at least 85%, at least 87%, at least 89%. at least 91%. at least 93%. at least 95%, at least 97% or at least 99% identity to the nucleotide sequence shown in SEQ ID NO: 7 and can function as the 3′ LTR. In some embodiments, the 3′ LTR sequence with the partially deleted U3 region comprises a nucleotide sequence that has at least 80% identity to the nucleotide sequence shown in SEQ ID NO: 7 and can function as the 3′ LTR. In other embodiments, the 3′ LTR sequence with the partially deleted U3 region comprises a nucleotide sequence that has at least 83% identity to the nucleotide sequence shown in SEQ ID NO: 7 and can function as the 3′ LTR. In some embodiments, the 3′ LTR sequence with the partially deleted U3 region comprises a nucleotide sequence that has at least 85% identity to the nucleotide sequence shown in SEQ ID NO: 7 and can function as the 3′ LTR. In other embodiments, the 3′ LTR sequence with the partially deleted U3 region comprises a nucleotide sequence that has at least 87% identity to the nucleotide sequence shown in SEQ ID NO: 7 and can function as the 3′ LTR. In some embodiments, the 3′ LTR sequence with the partially deleted U3 region comprises a nucleotide sequence that has at least 89% identity to the nucleotide sequence shown in SEQ ID NO: 7 and can function as the 3′ LTR. In other embodiments, the 3′ LTR sequence with the partially deleted U3 region comprises a nucleotide sequence that has at least 91% identity to the nucleotide sequence shown in SEQ ID NO: 7 and can function as the 3′ LTR. In some embodiments, the 3′ LTR sequence with the partially deleted U3 region comprises a nucleotide sequence that has at least 93% identity to the nucleotide sequence shown in SEQ ID NO: 7 and can function as the 3′ LTR. In other embodiments, the 3′ LTR sequence with the partially deleted U3 region comprises a nucleotide sequence that has at least 95% identity to the nucleotide sequence shown in SEQ ID NO: 7 and can function as the 3′ LTR. In some embodiments, the 3′ LTR sequence with the partially deleted U3 region comprises a nucleotide sequence that has at least 97% identity to the nucleotide sequence shown in SEQ ID NO: 7 and can function as the 3′ LTR. In other embodiments, the 3′ LTR sequence with the partially deleted U3 region comprises a nucleotide sequence that has at least 99% identity to the nucleotide sequence shown in SEQ ID NO: 7 and can function as the 3′ LTR. In a specific embodiment, the 3′ LTR sequence with the partially deleted U3 region comprises the nucleotide sequence shown in SEQ ID NO: 7. In another specific embodiment, the 3′ LTR sequence with the partially deleted U3 region is the nucleotide sequence shown in SEQ ID NO: 7.

In some embodiments, the vector further comprises a Blasticidin resistance gene sequence in place of a Puro (puromycin) resistance gene sequence. In some embodiments, the Blasticidin resistance gene sequence of the present invention comprises a nucleotide sequence shown in SEQ ID NO: 6 or a nucleotide sequence that has at least 80% identity to the nucleotide sequence shown in SEQ ID NO: 6 and has resistance. In other embodiments, the Blasticidin resistance gene sequence comprises a nucleotide sequence that has at least 80%, at least 83%, at least 85%, at least 87%, at least 89%, at least 91%, at least 93%, at least 95%. at least 97% or at least 99% identity to the nucleotide sequence shown in SEQ ID NO: 6 and has resistance. In some embodiments, the Blasticidin resistance gene sequence comprises a nucleotide sequence that has at least 80% identity to the nucleotide sequence shown in SEQ ID NO: 6 and has resistance. In other embodiments, the Blasticidin resistance gene sequence comprises a nucleotide sequence that has at least 83% identity to the nucleotide sequence shown in SEQ ID NO: 6 and has resistance. In some embodiments, the Blasticidin resistance gene sequence comprises a nucleotide sequence that has at least 85% identity to the nucleotide sequence shown in SEQ ID NO: 6 and has resistance. In other embodiments, the Blasticidin resistance gene sequence comprises a nucleotide sequence that has at least 87% identity to the nucleotide sequence shown in SEQ ID NO: 6 and has resistance. In some embodiments, the Blasticidin resistance gene sequence comprises a nucleotide sequence that has at least 89% identity to the nucleotide sequence shown in SEQ ID NO: 6 and has resistance. In other embodiments, the Blasticidin resistance gene sequence comprises a nucleotide sequence that has at least 91% identity to the nucleotide sequence shown in SEQ ID NO: 6 and has resistance. In some embodiments, the Blasticidin resistance gene sequence comprises a nucleotide sequence that has at least 93% identity to the nucleotide sequence shown in SEQ ID NO: 6 and has resistance. In other embodiments, the Blasticidin resistance gene sequence comprises a nucleotide sequence that has at least 95% identity to the nucleotide sequence shown in SEQ ID NO: 6 and has resistance. In some embodiments, the Blasticidin resistance gene sequence comprises a nucleotide sequence that has at least 97% identity to the nucleotide sequence shown in SEQ ID NO: 6 and has resistance. In other embodiments. the Blasticidin resistance gene sequence comprises a nucleotide sequence that has at least 99% identity to the nucleotide sequence shown in SEQ ID NO: 6 and has resistance. In a specific embodiment, the Blasticidin resistance gene sequence comprises the nucleotide sequence shown in SEQ ID NO: 6. In a specific embodiment, the Blasticidin resistance gene sequence is the nucleotide sequence shown in SEQ ID NO: 6.

The present invention specifically provides a lentiviral expression vector improved on the basis of PLVX-Puro, which is characterized in that the vector comprises the following improved sequences:

(1) a CMV enhancer sequence and a CMV promoter sequence upstream of 5′ LTR,
(2) a truncated PLVX-Puro 5′ LTR sequence,
(3) an EFS promoter sequence as a target gene promoter,
(4) a P2A linker sequence downstream of a multiple cloning site, and
(5) a 3′ LTR sequence with a partially deleted U3 region.

In some embodiments, the present invention provides the lentiviral expression vector improved on the basis of PLVX-Puro, which comprises the following improved sequences:

(1) a CMV enhancer sequence and a CMV promoter sequence upstream of 5′ LTR,
(2) a truncated PLVX-Puro 5′ LTR sequence,
(3) an EFS promoter sequence as a target gene promoter,
(4) a P2A linker sequence downstream of a multiple cloning site, and
(5) a Blasticidin resistance gene sequence in place of a Puro resistance gene sequence.

In some embodiments, the present invention provides the lentiviral expression vector improved on the basis of PLVX-Puro, which comprises the following improved sequences:

(1) a CMV enhancer sequence and a CMV promoter sequence upstream of 5′ LTR,
(2) a truncated PLVX-Puro 5′ LTR sequence,
(3) an EFS promoter sequence as a target gene promoter,
(4) a P2A linker sequence downstream of a multiple cloning site,
(5) a Blasticidin resistance gene sequence in place of a Puro resistance gene sequence, and
(6) a 3′ LTR sequence with a partially deleted U3 region.

In an embodiment of the present invention, in the lentiviral expression vector, the CMV enhancer sequence upstream of the 5′ LTR comprises a nucleotide sequence that has at least 80%, at least 85%, at least 87%, at least 89%, at least 91%, at least 93%, at least 95%, at least 97% or at least 99% identity to the nucleotide sequence shown in SEQ ID NO: 1 and has an enhancer function; the CMV promoter sequence upstream of the 5′ LTR comprises a nucleotide sequence that has at least 80%, at least 85%, at least 87%, at least 89%, at least 91%, at least 93%, at least 95%, at least 97% or at least 99% identity to the nucleotide sequence shown in SEQ ID NO: 2 and has a promoter function; the truncated 5′ LTR comprises a nucleotide sequence that has at least 80%, at least 85%. at least 87%, at least 89%, at least 91%, at least 93%. at least 95%, at least 97% or at least 99% identity to the nucleotide sequence shown in SEQ ID NO: 3 and can function as the 5′ LTR; the EFS promoter sequence comprises a nucleotide sequence that has at least 80%, at least 85%, at least 87%, at least 89%. at least 91%, at least 93%, at least 95%, at least 97% or at least 99% identity to the nucleotide sequence shown in SEQ ID NO: 4 and has a promoter activity; the P2A linker sequence comprises a nucleotide sequence that has at least 80%, at least 85%, at least 87%, at least 89%, at least 91%, at least 93%, at least 95%, at least 97% or at least 99% identity to the nucleotide sequence shown in SEQ ID NO: 5 and has a ligation function; the Blasticidin resistance gene sequence comprises a nucleotide sequence that has at least 80%, at least 85%, at least 87%, at least 89%, at least 91%, at least 93%, at least 95%, at least 97% or at least 99% identity to the nucleotide sequence shown in SEQ ID NO: 6 and has resistance: or/and the 3′ LTR sequence with the partially deleted U3 region comprises a nucleotide sequence that has at least 80% identity to the nucleotide sequence shown in SEQ ID NO: 7 and can function as the 3′ LTR. In other embodiments, in the lentiviral expression vector, the CMV enhancer sequence upstream of the 5′ LTR comprises the nucleotide sequence shown in SEQ ID NO: 1; the CMV promoter sequence upstream of the 5′ LTR comprises the nucleotide sequence shown in SEQ ID NO: 2; the truncated 5′ LTR comprises the nucleotide sequence shown in SEQ ID NO: 3: the EFS promoter sequence comprises the nucleotide sequence shown in SEQ ID NO: 4; the P2A linker sequence comprises the nucleotide sequence shown in SEQ ID NO: 5; the Blasticidin resistance gene sequence comprises the nucleotide sequence shown in SEQ ID NO: 6: or/and the 3′ LTR sequence with the partially deleted U3 region comprises the nucleotide sequence shown in SEQ ID NO: 7. In a specific embodiment, in the lentiviral expression vector, the CMV enhancer sequence upstream of the 5′ LTR is the nucleotide sequence shown in SEQ ID NO: 1: the CMV promote sequence is the nucleotide sequence shown in SEQ ID NO: 2; the truncated 5′ LTR is the nucleotide sequence shown in SEQ ID NO: 3; the EFS promoter sequence is the nucleotide sequence shown in SEQ ID NO: 4; the P2A linker sequence is the nucleotide sequence shown in SEQ ID NO: 5; the Blasticidin resistance gene sequence is the nucleotide sequence shown in SEQ ID NO: 6; or/and the 3′ LTR sequence with the partially deleted U3 region is the nucleotide sequence shown in SEQ ID NO: 7.

In another aspect, the present invention provides a method of improving a lentiviral expression vector on the basis of PLVX-Puro, the method comprising:

(1) adding a CMV promoter sequence to the upstream of 5′ LTR,
(2) replacing a CMV promoter of a target gene with an EFS promoter sequence, and
(3) deleting a PGK promoter downstream of a multiple cloning site and introducing a P2A linker sequence.

Further, the improvement method comprises the following one or more improving steps:

(1) adding a CMV enhancer sequence to the upstream of 5′ LTR,
(2) replacing the original 5′ LTR with a truncated 5′ LTR sequence, and
(3) replacing original 3′ LTR with a 3′ LTR sequence with a partially deleted U3 region.

In some embodiments, the present invention provides the method of improving the lentiviral expression vector on the basis of PLVX-Puro, which comprises:

(1) adding a CMV enhancer sequence and a CMV promoter sequence to the upstream of the 5′ LTR.
(2) replacing a CMV promoter of a target gene with an EFS promoter sequence, and
(3) deleting a PGK promoter downstream of a multiple cloning site and introducing a P2A linker sequence.

In other embodiments, the present invention provides the method of improving the lentiviral expression vector on the basis of PLVX-Puro, which comprises:

(1) adding a CMV enhancer sequence and a CMV promoter sequence to the upstream of the 5′ LTR.
(2) replacing a CMV promoter of a target gene with an EFS promoter sequence.
(3) deleting a PGK promoter downstream of a multiple cloning site and introducing a P2A linker sequence,
(4) replacing the original 5′ LTR with a truncated 5′ LTR sequence, and
(5) replacing the original 3′ LTR with a 3′ LTR sequence with a partially deleted U3 region.

Further, the method of improving the lentiviral expression vector of the present invention comprises replacing a Puro resistance gene with a Blasticidin resistance gene sequence.

In the method of improving the lentiviral vector provided by the present invention, the CMV promoter sequence upstream of the 5′ LTR comprises a nucleotide sequence that has at least 80%, at least 85%. at least 87%, at least 89%, at least 91%, at least 93%, at least 95%, at least 97% or at least 99% identity to the nucleotide sequence shown in SEQ ID NO: 2 and has a promoter activity, the EFS promoter sequence comprises a nucleotide sequence that has at least 80%, at least 85%, at least 87%, at least 89%, at least 91%, at least 93%, at least 95%, at least 97% or at least 99% identity to the nucleotide sequence shown in SEQ ID NO: 4 and has a promoter activity, or/and the P2A linker sequence comprises a nucleotide sequence that has at least 80%, at least 85%, at least 87%, at least 89%, at least 91%, at least 93%, at least 95%, at least 97% or at least 99% identity to the nucleotide sequence shown in SEQ ID NO: 5 and has a ligation function. In some embodiments, the CMV promoter sequence upstream of the 5′ LTR comprises a nucleotide sequence that has at least 80% identity to the nucleotide sequence shown in SEQ ID NO: 2 and has a promoter activity; the EFS promoter sequence comprises a nucleotide sequence that has at least 80% identity to a nucleotide sequence shown in SEQ ID NO: 4 and has a promoter activity; and/or the P2A linker sequence comprises a nucleotide sequence that has at least 80% identity to a nucleotide sequence shown in SEQ ID NO: 5 and has a ligation function. In other embodiments, the CMV promoter sequence upstream of the 5′ LTR comprises the nucleotide sequence shown in SEQ ID NO: 2; the EFS promoter sequence comprises the nucleotide sequence shown in SEQ ID NO: 4; and/or the P2A linker sequence comprises the nucleotide sequence shown in SEQ ID NO: 5. In a specific embodiment, the CMV promoter sequence upstream of the 5′ LTR is the nucleotide sequence shown in SEQ ID NO: 2; the EFS promoter sequence is the nucleotide sequence shown in SEQ ID NO: 4; and/or the P2A linker sequence is the nucleotide sequence shown in SEQ ID NO: 5.

In the method of improving the lentiviral vector provided by the present invention, the CMV enhancer sequence added comprises a nucleotide sequence that has at least 80%, at least 85%, at least 87%. at least 89%, at least 91%, at least 93%, at least 95%, at least 97% or at least 99% identity to the nucleotide sequence shown in SEQ ID NO: 1 and has an enhancer function; the truncated 5′ LTR sequence comprises a nucleotide sequence that has at least 80%, at least 85%, at least 87%, at least 89%, at least 91%, at least 93%, at least 95%, at least 97% or at least 99% identity to the nucleotide sequence shown in SEQ ID NO: 3 and can function as the 5′ LTR; and/or the 3′ LTR sequence with the partially deleted U3 region comprises a nucleotide sequence that has at least 80%, at least 85%, at least 87%, at least 89, at least 91%, at least 93/a, at least 95%, at least 97% or at least 99% identity to the nucleotide sequence shown in SEQ ID NO: 7 and can function as the 3′ LTR. In some embodiments, the CMV enhancer sequence added comprises a nucleotide sequence that has at least 80% identity to the nucleotide sequence shown in SEQ ID NO: 1 and has an enhancer function; the truncated 5′ LTR sequence comprises a nucleotide sequence that has at least 80% identity to a nucleotide sequence shown in SEQ ID NO: 3 and can function as the 5′ LTR; and/or the 3′ LTR sequence with the partially deleted U3 region comprises a nucleotide sequence that has at least 80% identity to the nucleotide sequence shown in SEQ ID NO: 7 and can function as the 3′ LTR. In other embodiments, the CMV enhancer sequence added comprises the nucleotide sequence shown in SEQ ID NO: 1; the truncated 5′ LTR sequence comprises the nucleotide sequence shown in SEQ ID NO: 3; and/or the 3′ LTR sequence with the partially deleted U3 region comprises the nucleotide sequence shown in SEQ ID NO: 7. In a specific embodiment, the CMV enhancer sequence added is the nucleotide sequence shown in SEQ ID NO: 1; the truncated 5′ LTR sequence is the nucleotide sequence shown in SEQ ID NO: 3; and/or the 3′ LTR sequence with the partially deleted U3 region is the nucleotide sequence shown in SEQ ID NO: 7.

Further, in the improvement method provided by the present invention, the Blasticidin resistance gene sequence comprises a nucleotide sequence that has at least 80%, at least 85%. at least 87%, at least 89%, at least 91%, at least 93%. at least 95%, at least 97% or at least 99% identity to the nucleotide sequence shown in SEQ ID NO: 6 and has resistance. In some embodiments, the Blasticidin resistance gene sequence comprises a nucleotide sequence that has at least 80% identity to the nucleotide sequence shown in SEQ ID NO: 6 and has resistance.

The present invention further provides a eukaryotic cell transfected with the lentiviral expression vector of the present invention. In some embodiments, the eukaryotic cell is a 293 cell, more preferably a 293T cell or a 293F cell. In other embodiments, the eukaryotic cell is a 293T cell. In other embodiments, the eukarvotic cell is a 293F cell.

The improved lentiviral expression vector or the eukaryotic cell transfected with the improved lentiviral expression vector of the present invention is used in the preparation of lentiviral particles.

In the present invention, by means of conventional technical means in the art, lentiviral particles can be prepared by using an improved lentiviral vector, including a second-generation, third-generation or fourth-generation lentiviral system. especially a third-generation lentiviral system which comprises an improved lentiviral expression vector, a packaging plasmid and an envelope plasmid. 293T or 293F cells are co-transfected, and the culture supernatant is collected to obtain lentiviral particles. The lentiviral particles are packaged and then used to transfect target cells. The improved lentiviral expression vector for transfection of the present invention is mixed and incubated with packing mix plasmids, and then the mixture is used to transfect 293F cells. 72 h after culture, the virus supernatant is collected, filtered and concentrated to obtain a virus suspension that can be used for determination of the virus functional titer and the physical titer and can also be used for transfection of target cells.

The use of the lentiviral expression vector of the present invention or the eukaryotic cell transfected with the lentiviral expression vector in the expression of an exogenous target gene comprises cloning the target gene into a multiple cloning site of the lentiviral expression vector to achieve the stable expression of the target gene. The coding region of the exogenous target gene of interest is constructed on the multiple cloning site of the lentiviral expression vector of the present invention to obtain a recombinant vector expressing the target gene. The recombinant vector, the lentiviral packaging vector and the envelope vector are used to co-transfect 293T or 293F cells for lentiviral packaging, and the packaged lentiviral particles act on target cells, so as to effectively express the exogenous gene. Fluorescence tracing and drug resistance screening of target cells are performed by using EGFP, a Puromycin (Puro) resistance gene and/or a Blasticidin resistance gene expressed by the lentiviral expression vector, so as to effectively screen and purify the target cells. In some embodiments, the target gene sequence is amplified and ligated with the linearized lentiviral expression vector of the present invention. The ligated product is transformed into JM108 competent cells and cultured in a plate containing resistance. A single colony is picked for culture and extracted to obtain a lentiviral expression vector containing the target gene. Further, the lentiviral expression vector containing the target gene for transfection is mixed and incubated with packaging mix plasmids, and then the mixture is used to transfect 293F cells. 72 h after culture, the virus supernatant is collected, filtered and concentrated to obtain a virus suspension. By means of adjustment, deletion and modification of some elements of the lentiviral expression vector of the present invention, the carrying capacity of the vector is greatly increased, which allows for packaging genes longer than 5 kb and obtaining a higher titer. For example, the vector can be used to package a Cas9 gene (4698 bps), an EGFR gene (4104 bps), a Cas9 and EGFP fusion protein gene (5349 bps), etc.

The improved lentiviral expression vector or the eukaryotic cell transfected with the lentiviral expression vector of the present invention is used for preparing a gene or cell therapy drug. Insertion of an exogenous target gene into a lentiviral expression vector can be applied to the preparation of a gene therapy drug or a cell therapy drug.

As used in the present invention, the term “lentivirus” refers to a complex group (or genus) of retroviruses. Illustrative lentiviruses include, but are not limited to: a human immunodeficiency virus (HIV, including HIV type 1 and HIV type 2); a visna-macdi virus (VMV); a caprine arthritis encephalitis virus (CAEV); an equine infectious anemia virus (EIAV): a feline immunodeficiency virus (FIV); a bovine immunodeficiency virus (BIV); and a simian immunodeficiency virus (SIV). In some embodiments, the HIV-based vector backbone is preferred.

The term “vector” used herein refers to a nucleic acid molecule capable of transferring or delivering another nucleic acid molecule. The transferred nucleic acid is typically ligated to (e.g., inserted into) the vector nucleic acid molecule. The vector may comprise sequences that direct autonomous replication in a cell, or may comprise sequences sufficient to allow for integration into DNA of the host cell. Useful vectors comprise, for example, plasmids (e.g., DNA plasmids or RNA plasmids), transposons, cosmids, bacterial artificial chromosomes and viral vectors. Useful viral vectors comprise, for example, replication-defective retroviruses and lentiviruses.

As will be apparent to those skilled in the art, the term “viral vector” is commonly used to refer to a nucleic acid molecule (e.g., a transfer plasmid) that includes a virus-derived nucleic acid element that typically facilitates transfer or integration of the nucleic acid molecule into the genome of a cell, or refer to a viral particle that mediates nucleic acid transfer. In addition to the nucleic acid, the viral particle will typically comprise various viral components and sometimes host cell components. The viral vector and the transfer plasmid contain structural and/or functional genetic elements derived primarily from viruses.

The terms “lentiviral vector” and “lentiviral expression vector” may be used to refer to lentiviral transfer plasmids and/or infectious lentiviral particles, and retroviral vectors or plasmids or parts thereof (including LTR) containing structural and functional genetic elements derived primarily from lentiviruses. Where elements such as cloning sites, promoters, regulatory elements and exogenous target genes are referred to herein, it should be understood that the sequences of these elements are present in the lentiviral particles of the present invention in the form of RNA and in the DNA plasmid of the present invention in the form of DNA.

At each end of a provirus is a structure called a“long terminal repeat” or “LTR”. The term “long terminal repeat (LTR)” refers to a base pair domain located at the end of retroviral DNA, which in its native sequence is a direct repeat and contains U3, R and U5 regions. The LTR generally provides functions important for retroviral gene expression (e.g., initiation, start, and polyadenylation of gene transcripts) and viral replication. The LTR contains numerous regulatory signals, including transcriptional control elements, polyadenylation signals, and sequences required for replication and integration of the viral genome. The viral LTR is divided into three regions called U3. R and U5. The U3 region contains enhancer and promoter elements. The U5 region is a sequence between the primer binding site and the R region and contains a polyadenylation sequence. The U3 and U5 regions are flanked by the R (repeat) region. The LTR consists of the U3, R and U5 regions and is present at the 5′ and 3′ ends of the viral genome. Adjacent to the 5′ LTR are sequences required for reverse transcription of the genome (a tRNA primer binding site) and for efficient packaging of the viral RNA into particles (a Ψ site).

Terms describing the orientation of a polynucleotide include: 5′ (usually referring to the end of the polynucleotide with a free phosphate group) and 3′ (usually referring to the end of the polynucleotide with a free hydroxyl (OH) group). The polynucleotide sequence can be annotated in the 5′ to 3′ orientation or 3′ to 5′ orientation.

The term “promoter” refers to the recognition site of a polynucleotide (DNA or RNA) to which the RNA polymerase binds. The term “enhancer” refers to a segment of DNA that contains a sequence capable of providing enhanced transcription and, in some cases, can function independently of its orientation relative to another control sequence. The enhancer can function cooperatively or additively with a promoter and/or other enhancer elements. The term “promoter/enhancer” refers to a segment of DNA that contains sequences capable of providing promoter and enhancer functions.

The term “packaging vector” refers to an expression vector or a viral vector that lacks a packaging signal but contains polynucleotides encoding one, two, three, four or more viral structural genes and/or accessory genes. Typically, the packaging vector is included in a packaging cell and introduced into a cell by means of transfection, transduction or infection. Methods for transfection, transduction or infection are well known to those skilled in the art. The retroviral/lentiviral transfer vectors encompassed in particular embodiments can be introduced into packaging cell lines by means of transfection. transduction or infection to generate producer cells or cell lines. The packaging vector can be introduced into human cells or cell lines by standard methods including, for example, calcium phosphate transfection, liposome transfection or electroporation. In some embodiments, the packaging vector is introduced into cells together with a dominant selectable marker such as neomycin, hygromycin, puromycin, blasticidin, bleomycin, a thymidine kinase, DHFR, a Gin synthase or ADA, and clones are then selected and isolated in the presence of appropriate drugs. The selectable marker gene can be ligated with a genetic entity encoded by the packaging vector, e.g., by an IRES or a self-cleaving viral peptide.

The term “transfection” refers to the transfer of a polynucleotide or other biologically active compounds from outside a cell into a cell such that the polynucleotide or biologically active compound is functional. Examples of transfection reagents for in-vitro delivery of polynucleotides to cells include, but are not limited to: liposomes, lipids, polyamines, calcium phosphate precipitates, histones, polyaziridines and amphoteric polyelectrolyte complexes and combinations thereof. Many in-vitro transfection reagents are cationic, which allows the reagents to bind or form a complex with negatively charged nucleic acids by means of electrostatic interactions.

The term “target gene” refers to a polynucleotide that encodes a polypeptide (i.e., a target polypeptide) and is inserted into an expression vector for desired expression. The vector may contain 1, 2, 3, 4, 5, 6, 7, 8, 9 or 10 target polynucleotides. The target polynucleotides and polypeptides encoded thereby include polynucleotides encoding wild-type polypeptides, as well as functional variants and fragments thereof. In specific embodiments, the functional variant has at least 80%, at least 90%, at least 95% or at least 99% identity to the corresponding wild-type reference polynucleotide or polypeptide sequence. In certain embodiments, the functional variant or fragment has at least 50%, at least 60%, at least 70%, at least 80% or at least 90% of the biological activity of the corresponding wild-type polypeptide.

Where reference is made to the sequence, the term “sequence identity” (also referred to as “Sequence Identity”) refers to the amount of identity, generally expressed as a percentage, between two sequences (e.g., a query sequence and a reference sequence). Typically, sequence alignment is performed and a gap (if any) is introduced before calculating the percentage identity between two sequences. If bases or amino acids in the two sequences are the same at a certain alignment position, the two sequences are considered to be identical or matched at that position. If the bases or amino acids in the two sequences are different, the two sequences are considered to be non-identical or mismatched at that position. In some algorithms, the number of matched positions is divided by the total number of positions in the alignment window to obtain sequence identity. In other algorithms, the number of gaps and/or the gap length are also taken into account. For the purpose of the present invention, the publicly available alignment software BLAST (available at ncbi.nlm.nih.gov) can be employed to obtain the optimal sequence alignment and calculate the sequence identity between two sequences by using default settings.

The basic backbone of the vector is derived from the PLVX-Puro lentiviral expression vector. This vector is a second-generation traditional lentiviral expression vector with limited carrying capacity. The improvement of the present invention comprises the following steps:

a. adding a CMV enhancer and a CMV promoter to the upstream of 5′ LTR of a traditional vector;
b. replacing the 5′ LTR of the traditional vector with an improved LTR, wherein the improved LTR is 181 bases in length, which is 454 bases shorter than the traditional one:
c. replacing the promoter of a target gene with EFS, which is 347 bases less than the traditional CMV promoter and is more suitable for CMV-silenced cells (e.g., blood suspension cells) to initiate transcription;
d. changing a double-promoter mode to a P2A linker ligation mode, which can greatly reduce the length of the plasmid backbone and the competition of different promoters in the vector for transcription factors;
e. replacing a resistance gene with a shorter Blasticidin; and
f. replacing the traditional redundant 3′ LTR with a truncated 3′ LTR.

Compared with the traditional lentiviral expression vector, the lentiviral expression vector improved by the present technique has the following advantages:

(1) the vector backbone is smaller, and the upper limit of virus packaging is larger, from the original exogenous gene carrying capacity of 3 kb to 5 kb; (2) the resulting virus titer is higher and the protein expression is stronger; (3) some replication elements are removed, such as the removal of part of the U3 region of the 3′ LTR, so that the safety is higher; and (4) compared with multiple promoters, a single promoter reduces the competition for transcription factors.

### BRIEF DESCRIPTION OF THE DRAWINGS

FIGS. 1A and 1B are an original Plvx-Puro lentiviral expression vector and an improved lentiviral expression vector, respectively.

FIGS. 2A to 2E show maps of expression vectors into which target genes EGFR, Cas9, Cas9+EGFP, CCR2+Cas9+EGFP and EGFR+Cas9 are inserted, respectively.

FIGS. 3A to 3C are respectively experimental result diagrams of fluorescence expression of vectors into which target genes (EGFR, Cas9+EGFP, and CCR2+EGFP+Cas9) in different lengths are inserted, in cells, as detected by FACS.

### DETAILED DESCRIPTION OF EMBODIMENTS

The embodiments of the present invention are described below by particular specific examples, and those skilled in the art can easily understand other advantages and effects of the present invention from the disclosure of the specification. The present invention can also be implemented or applied by other different specific embodiments, and various details in the specification can also be modified or altered on the basis of different viewpoints and applications without departing from the spirit of the present invention.

Before the specific embodiments of the present invention are further described, it should be understood that the scope of protection of the present invention is not limited to the following particular specific embodiments. It should also be understood that the terms used in the examples of the present invention are intended to describe the particular specific embodiments, rather than limiting the scope of protection of the present invention, and have meanings commonly understood by those skilled in the art. In the specification and claims of the present invention, unless otherwise clearly stated in the context, the singular forms “a”, “an”, and “the” include the plural forms.

When numerical ranges are given in the examples, it should be understood that unless otherwise indicated in the present invention, the two endpoints of each numerical range and any value between the two endpoints can be selected. Unless otherwise defined, all technical and scientific terms used in the present invention have the same meanings as commonly understood by those skilled in the art. In addition to the specific methods, devices, and materials used in the examples, according to the knowledge in the prior art and the disclosure of the present invention, those skilled in the art can also use any prior art methods, devices, and materials which are similar or equivalent to the methods, devices, and materials described in the examples of the present invention to realize the present invention.

### Example 1. Construction of Improved Lentiviral Expression Vector Backbone

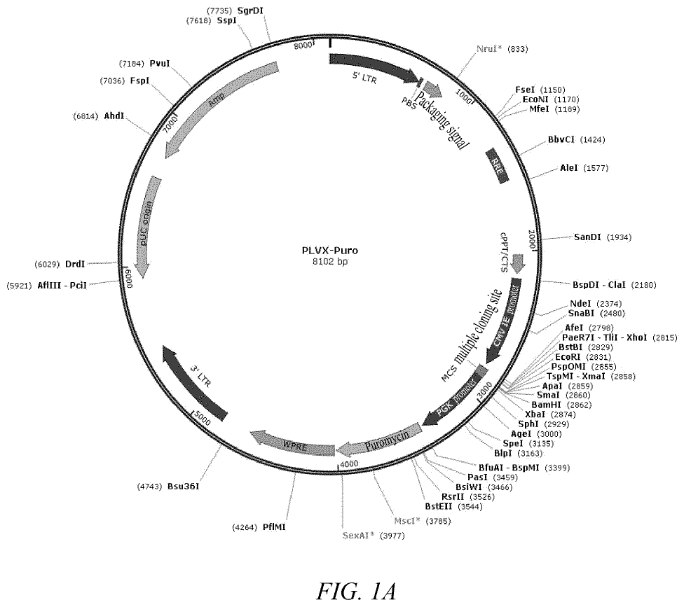

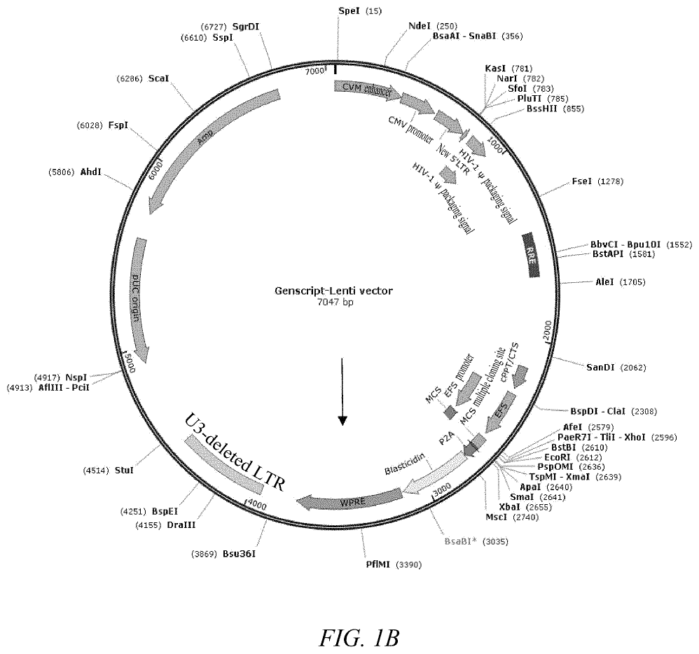

The plasmid backbone of the present invention is based on PLVX-Puro (Takara, 632164, see the map of FIG. 1A), and is improved via the following steps:

a. adding a CMV enhancer (SEQ ID NO: 1) and promoter (SEQ ID NO: 2) to the upstream of 5′ LTR by means of gene synthesis and subcloning;
b. replacing the original 5′ LTR with a shorter new 5′ LTR (SEQ ID NO: 3) of which the length is 181 bps;
c. replacing the CMV promoter on an original vector with a broader-spectrum and shorter EFS promoter (SEQ ID NO: 4) of which the length is 256 bps:
d. deleting a PGK promoter downstream of a multiple cloning site of the original vector and, at the same time, introducing a P2A linker sequence (SEQ ID NO: 5);
e. replacing original resistance gene against Puro with a shorter resistance gene against Blasticidin (SEQ ID NO: 6) of which the length is 399 bps; and
f. replacing original 3′ LTR with an LTR (SEQ ID NO: 7) with a partially deleted U3 in order to obtain higher safety.

The specific experimental steps were as follows:

### Vector-Improving Steps:

1.1 A CMV enhancer, a CMV promoter and a new 5′LTR sequence were amplified to obtain a PCR product. The PCR product was subjected to electrophoresis detection, a band having a size consistent with the expected size of the target sequence was cut off, and the product was recovered for ligation and transformation. The amplified PCR product above was ligated with PLVX-puro linearized with PvuI/MfeI (PvuI: NEB, R3150GS; MfeI: NEB, R3589S) by using GenBuilder (GenScript, M00712) to obtain a ligated product. The ligated product was transformed into TOP10 (GenScript, NA) competent cells by a thermal shock method, the transformed product was evenly spread on an LB plate culture medium (GenScrinpt, NA) containing ampicillin resistance with a spreader, and the plate was inversely placed in a 37° C. incubator for overnight culture. A single colony was picked with a sterile pipette tip as a PCR template for colony screening, and transferred to an LB liquid culture medium (GenScript, NA) containing ampicillin resistance for culture. The PCR product for bacterial detection was subjected to electrophoresis detection, and a clone having a band with the size consistent with the expected size was selected for bacterial shaking and plasmid extraction.

1.2 An EFS promoter sequence was amplified to obtain a PCR product. The PCR product was subjected to electrophoresis detection, a band having a size consistent with the expected size of the target sequence was cut off, and the product was recovered for ligation and transformation. The amplified PCR product above was ligated with the plasmid obtained in step 1.1 which is linearized with ClaI/XhoI (ClaI: NEB, R0197V; XhoI: NEB, R0146L) by using GenBuilder (GenScript, M00712) to obtain a ligated product. The ligated product was transformed into TOP10 (GenScript, NA) competent cells by a thermal shock method, the transformed product was evenly spread on an LB plate culture medium (GenScript, NA) containing ampicillin resistance with a spreader, and the plate was inversely placed in a 37° C. incubator for overnight culture. A single colony was picked with a sterile pipette tip as a PCR template for colony screening, and transferred to an LB liquid culture medium (GenScript, NA) containing ampicillin resistance for culture. The PCR product for bacterial detection was subjected to electrophoresis detection, and a clone having a band with the size consistent with the expected size was selected for bacterial shaking and plasmid extraction.

1.3 A P2A sequence was amplified to obtain a PCR product. The PCR product was subjected to electrophoresis detection, a band having a size consistent with the expected size of the target sequence was cut off, and the product was recovered for ligation and transformation. The amplified PCR product above was ligated with the plasmid obtained in step 1.2 which is linearized with XbaI/BSiW (XbaI: NEB, R0145S; BSiW: NEB, R3553S) by using GenBuilder (GenScript, M00712) to obtain a ligated product. The ligated product was transformed into TOP10 (GenScript, NA) competent cells by a thermal shock method, the transformed product was evenly spread on an LB plate culture medium (GenScript, NA) containing ampicillin resistance with a spreader, and the plate was inversely placed in a 3T′C incubator for overnight culture. A single colony was picked with a sterile pipette tip as a PCR template for colony screening, and transferred to an LB liquid culture medium (GenScript, NA) containing ampicillin resistance for culture. The PCR product for bacterial detection was subjected to electrophoresis detection, and a clone having a band with the size consistent with the expected size was selected for bacterial shaking and plasmid extraction.

1.4 A Blasticidin sequence was amplified to obtain a PCR product. The PCR product was subjected to electrophoresis detection, a band having a size consistent with the expected size of the target sequence was cut off, and the product was recovered for ligation and transformation. The amplified PCR product above was ligated with the plasmid obtained in step 1.3 which is linearized with BSU36I/XbaI (BSU36I: NEB, R0524V; XbaI: NEB, R0145S) by using GenBuilder (GenScript, M00712) to obtain a ligated product. The ligated product was transformed into TOP10 (GenScript, NA) competent cells by a thermal shock method, the transformed product was evenly spread on an LB plate culture medium (GenScript. NA) containing ampicillin resistance with a spreader, and the plate was inversely placed in a 37° C. incubator for overnight culture. A single colony was picked with a sterile pipette tip as a PCR template for colony screening, and transferred to an LB liquid culture medium (GenScript, NA) containing ampicillin resistance for culture. The PCR product for bacterial detection was subjected to electrophoresis detection, and a clone having a band with the size consistent with the expected size was selected for bacterial shaking and plasmid extraction.

1.5 A 3′ LTR sequence was amplified to obtain a PCR product. The PCR product was subjected to electrophoresis detection, a band having a size consistent with the expected size of the target sequence was cut off, and the product was recovered for ligation and transformation. The amplified PCR product above was ligated with the plasmid obtained in step 1.4 which is linearized with BSU36I/StuI (BSU361: NEB, R0524V; StuI: NEB, R0187S) by using GenBuilder (GenScript, M00712) to obtain a ligated product. The ligated product was transformed into TOP10 (GenScript, NA) competent cells by a thermal shock method, the transformed product was evenly spread on an LB plate culture medium (GenScript. NA) containing ampicillin resistance with a spreader, and the plate was inversely placed in a 37° C. incubator for overnight culture. A single colony was picked with a sterile pipette tip as a PCR template for colony screening, and transferred to an LB liquid culture medium (GenScript. NA) containing ampicillin resistance for culture. The PCR product for bacterial detection was subjected to electrophoresis detection, and a clone having a band with the size consistent with the expected size was selected for bacterial shaking and plasmid extraction.

By means of the improvement for PLVX-Puro above, an improved lentiviral expression vector (Genscript-Lenti vector) is obtained, and the map is as shown in FIG. 1B.

### Example 2. Verification of Gene Expression Function of Improved Lentiviral Expression Vector

### 2.1 Construction of Improved Lentiviral Expression Vector

In the experiment, five genes with different lengths as shown in Table 1 were selected, and each gene was subcloned into the improved lentiviral expression vector backbone in Example 1.

The steps of subcloning different target genes were as follows:

(1) An EGFR gene sequence (as shown in SEQ ID NO: 8) was amplified to obtain a PCR product. The PCR product was subjected to electrophoresis detection, a band having a size consistent with the expected size of the target sequence was cut off, and the product was recovered for ligation and transformation. The amplified PCR product above was ligated with the vector (Genscript-Lenti vector) improved in Example 1 which is linearized with ClaI/SaCII (ClaI: NEB, R0197V; SaCII: NEB, R0157S) by using GenBuilder (GenScript, M00712) to obtain a ligated product. The ligated product was transformed into JM108 (GenScript, NA) competent cells by a thermal shock method, the transformed product was evenly spread on an LB plate culture medium (GenScript, NA) containing ampicillin resistance with a spreader, and the plate was inversely placed in a 37° C. incubator for overnight culture. A single colony was picked with a sterile pipette tip as a PCR template for colony screening, and transferred to an LB liquid culture medium (GenScript, NA) containing ampicillin resistance for culture. The PCR product for bacterial detection was subjected to electrophoresis detection, and a clone having a band with the size consistent with the expected size was selected for bacterial shaking and plasmid extraction.

(2) A Cas9 gene sequence (as shown in SEQ ID NO: 9) was amplified to obtain a PCR product. The PCR product was subjected to electrophoresis detection, a band having a size consistent with the expected size of the target sequence was cut off, and the product was recovered for ligation and transformation. The amplified PCR product above was ligated with the vector (Genscript-Lenti vector) improved in Example 1 which is linearized with XhoII/XbaI (XhoI: NEB, R0146L; XbaI: NEB, R0145S) by using GenBuilder (GenScript, M00712) to obtain a ligated product. The ligated product was transformed into JM108 (GenScript, NA) competent cells by a thermal shock method, the transformed product was evenly spread on an LB plate culture medium (GenScript, NA) containing ampicillin resistance with a spreader, and the plate was inversely placed in a 37° C. incubator for overnight culture. A single colony was picked with a sterile pipette tip as a PCR template for colony screening, and transferred to an LB liquid culture medium (GenScript, NA) containing ampicillin resistance for culture. The PCR product for bacterial detection was subjected to electrophoresis detection, and a clone having a band with the size consistent with the expected size was selected for bacterial shaking and plasmid extraction.

(3) An EGFP gene sequence (as shown in SEQ ID NO: 12) was amplified to obtain a PCR product. The PCR product was subjected to electrophoresis detection, a band having a size consistent with the expected size of the target sequence was cut off, and the product was recovered for ligation and transformation. The amplified PCR product above was ligated with the vector obtained in step (2) which is linearized with BamHI (BamHI: NEB, R3136V) by using GenBuilder (GenScript, M00712) to obtain a ligated product. The ligated product was transformed into JM108 (GenScript. NA) competent cells by a thermal shock method, the transformed product was evenly spread on an LB plate culture medium (GenScript, NA) containing ampicillin resistance with a spreader, and the plate was inversely placed in a 37° C. incubator for overnight culture. A single colony was picked with a sterile pipette tip as a PCR template for colony screening, and transferred to an LB liquid culture medium (GenScript, NA) containing ampicillin resistance for culture. The PCR product for bacterial detection was subjected to electrophoresis detection, and a clone having a band with the size consistent with the expected size was selected for bacterial shaking and plasmid extraction.

(4) A CCR2 gene sequence (as shown in SEQ ID NO: 10) was amplified to obtain a PCR product. The PCR product was subjected to electrophoresis detection, a band having a size consistent with the expected size of the target sequence was cut off, and the product was recovered for ligation and transformation. The amplified PCR product above was ligated with the vector obtained in step (3) which is linearized with AfeI (AfeI: NEB, R0652L) by using GenBuilder (GenScript, M00712) to obtain a ligated product. The ligated product was transformed into JM108 (GenScript, NA) competent cells by a thermal shock method, the transformed product was evenly spread on an LB plate culture medium (GenScript, NA) containing ampicillin resistance with a spreader, and the plate was inversely placed in a 37° C. incubator for overnight culture. A single colony was picked with a sterile pipette tip as a PCR template for colony screening, and transferred to an LB liquid culture medium (GenScript, NA) containing ampicillin resistance for culture. The PCR product for bacterial detection was subjected to electrophoresis detection, and a clone having a band with the size consistent with the expected size was selected for bacterial shaking and plasmid extraction.

TABLE 1

| | Length of fragments |
|---|---|
| Name of genes | (including P2A and |
| expressed by the | Blasticidin) inserted to the |
| vector | MCS site |
| EGFR | 4104 bps |
| Cas9 | 4698 bps |
| Cas9 + EGFP | 5349 bps |
| CCR2 + Cas9 + | 6471 bps |
| EGFP | |
| EGFR + Cas9 | 8256 bps |

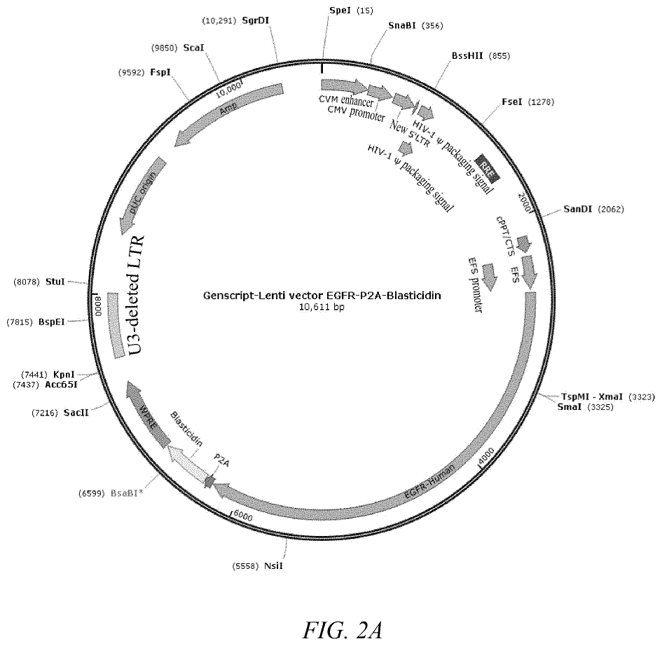

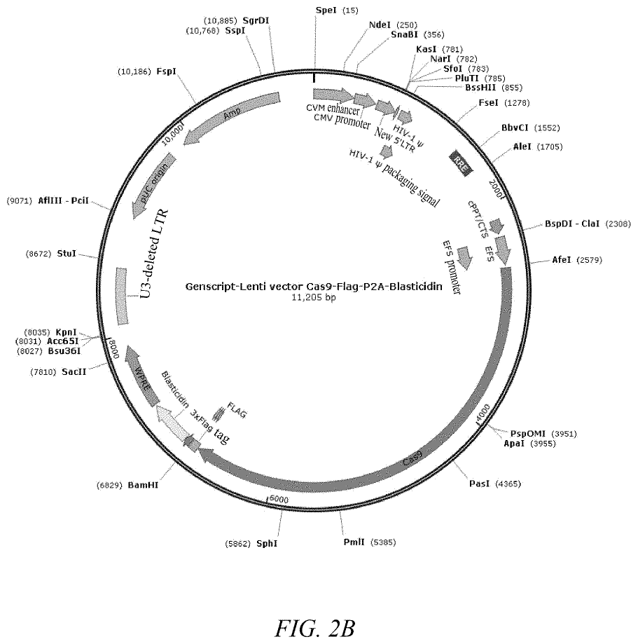

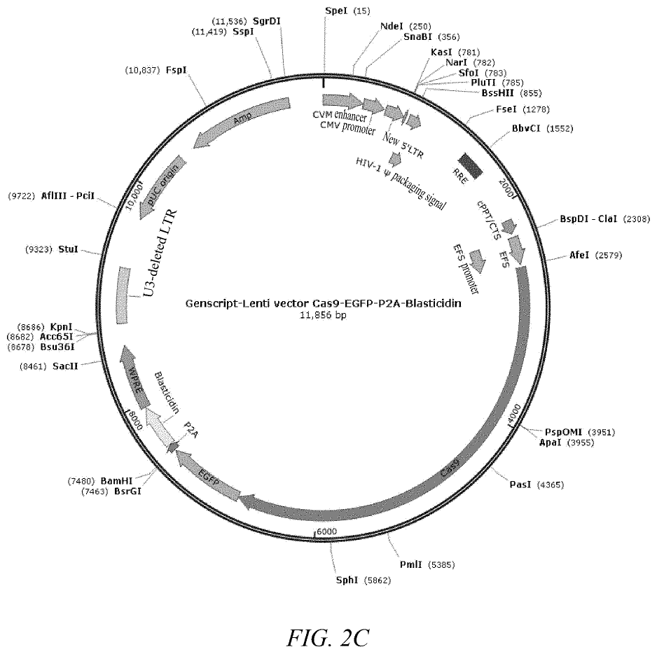

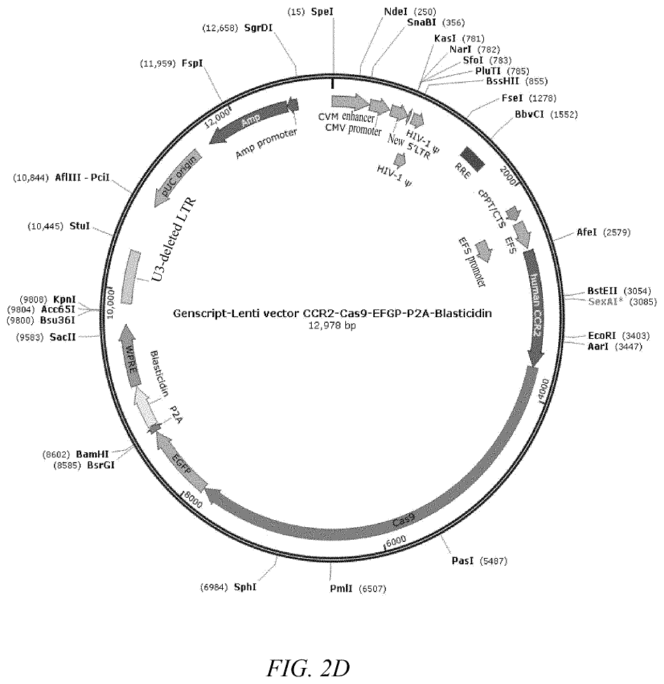

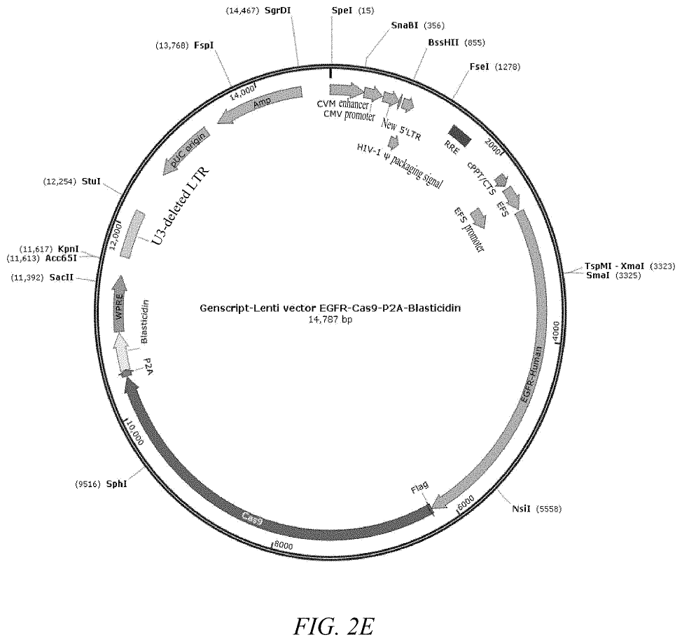

The maps of the constructed test vectors are shown in FIGS. 2A to 2E, respectively.

### 2.2 Cell Preparation

1) 24 h before lentiviral packaging, suspended 293F cells were inoculated in a 125 mL triangular flask at a density of (3.5-4)×10⁶ cells/mL;

2) the cell culture flask was placed in a 37° C., 5% CO₂ incubator for culture, and the rotating speed of the shaker was adjusted to 125 rpm, followed by overnight culture; and

3) the cell viability and the number of cells were measured the next day, and when the cell viability reached 90% or above and the total number of cells reached 7×10⁹, the next step was performed.

### 2.3 Plasmid Transfection

1) According to the experimental requirements, 125 mL shake flasks are prepared for different groups (see Table 2 for grouping), and transfection was carried out simultaneously under the same conditions;

2) the transfection method was as follows: on the day of virus packaging, cells were observed and measured for the viability, and if the cells were bright and round and had a viability greater than 90%. the lentiviral packaging could be performed; according to the density of 3.5×10⁶ cells/mL, cell fluid having a volume V₁ was taken; 5% Vₜₒₜₐₗ LV-MAX™ Supplement (Gibco, A35348) was added; a (85% Vₜₒₜₐₗ−V₁) fresh culture medium (Thermo, A3583401) was added, and then transferred to a C02 incubator for use. Plasmids were taken out and thawed, and then transfection was carried out according to the following procedure: DNA diluent was prepared, to which packaging mix plasmids (Thermo, A43237) at 1.5 ug/ml relative to the total volume, then target plasmids at 1 ug/ml relative to the total volume, and then Opti-MEM with a ratio of 5% relative to the total volume were added, and the mixture was mixed uniformly and placed at room temperature. Transfection reagent diluent was prepared, to which Opti-MEM with a ratio of 5% relative to the total volume, and then an LV-MAX transfection reagent at 1.5 pg/ml relative to the total volume were added, and the mixture was mixed uniformly and incubated at room temperature for 1 min. The DNA diluent and the transfection reagent diluent were mixed uniformly, and the mixture was incubated at room temperature for 10 min, then added to a shake flask and shaken gently. The culture flask was put back into the incubator and cultured for additional 5-6 hours; after transfection, the cells were cultured at 37° C. in 5% CO2. The rotating speed of the shaker (Kylin-Bell, Camel Shaker Model TS-2) was set to be 125 rpm; 4% Voaai LV-MAX™ Enhancer (Gibco, A35348) was added after 5-6 hours, and the shake flask was put back into the incubator and continued to be cultured.

### 2.4 Virus Collection

1) The virus supernatant was collected 72 h after transfection;

2) after the virus supernatant was obtained, the supernatant was first subjected to centrifugation in a centrifuge (Eppendorf, 5810R) at 4° C. and 1000-3000 rpm for 10 min, and then transferred to a new centrifuge tube and filtered with a 0.45-micron filter membrane (Millipore. Cat. No. SLHP033RB);

3) the obtained supernatant was mixed uniformly with a concentrated reagent (40% PEG8000, 4% sodium chloride) in a ratio of 4:1, and then left overnight at 4° C.; the next day, the concentrated virus solution was centrifuged at 4° C. and 1500 g for 45 min to 1 h; the supernatant was discarded, and an appropriate amount of 4° C. pre-cooled DPBS (Gibco, Cat. No. 14190-144) was added, the precipitate was suspended by means of gentle and full pipetting to obtain a virus suspension, which is subpackaged and stored in a −80° C. refrigerator.

### 2.5 Determination of Virus Titer by p24 Method

A commercial kit Lenti-X™ p24 Rapid Titer Kit (Takara, Cat. No. 632200) was used for titer determination, and 50 μL of virus suspension was taken for detection. For specific operations, reference was made to the kit instructions. A microplate reader was used for determination (the absorbance value of the sample was detected at an OD of 450 nm), and the data was imported into the template for data processing. The results are as shown in Table 2.

The calculation formula is as follows:

1 virus particle is approximately equal to 8×10⁻⁵ pg p24;
1 ng p24 is approximately equal to about 1.25×10⁷ viral particles:
1 IFU is approximately equal to 100 to 1000 virus particles, and the median is taken to calculate the virus titer.

TABLE 2

| Name of | Inserted fragments | Titer value (× |
|---|---|---|
| inserted | (including P2A and | 10\hat{ }6 |
| genes | Blasticidin) | IFU/mL) |
| EGFR | 4104 bps | 147 |
| Cas9 | 4698 bps | 135 |
| Cas9 + EGFP | 5349 bps | 86 |
| CCR2 + EGFP + | 6471 bps | 47 |
| Cas9 | | |
| EGFR + Cas9 | 8280 bps | 68 |

The virus titer as calculated in Table 2 shows that the packaging capacity of the improved lentiviral expression vector of the present invention can exceed the upper limit (3 kb) of the traditional expression vector; and the improved expression vector can achieve a high titer for genes longer than 5 kb.

### Example 3. Determination of Functional Titer of Improved Lentiviral Expression Vector

With reference to the method of Example 2.1. some of the long-fragment genes (see Table 3) were selected and subcloned into the improved lentiviral expression vector backbone. In addition, with reference to the determination method in Example 2.5. the p24 physical titer determination and the FACS functional titer test were carried out.

TABLE 3

| Name of inserted | Length of inserted |
|---|---|
| genes | fragments |
| EGFR | 4104 bps |
| Cas9 | 4698 bps |
| Cas9 + EGFP | 5349 bps |
| CCR2 + EGFP + | 6471 bps |
| Cas9 | |

The experimental steps of the FACS functional test were as follows:

(1) 293T cells were plated on a 96-well plate and cultured overnight, so that the cell confluence rate fell within the range of 30% to 50% and the initial cell concentration was approximately 3×10⁵/mL before transfection;
2) the virus stock solution was diluted according to the virus concentration gradient (10⁻¹ to 10⁻⁶) in Table 4;
3) the culture medium from the 96-well plate was removed, and 1 mL of the culture medium (DMEM+10% FBS) containing viruses diluted in gradients and Polybrene was added to the 96-well plate containing cells, followed by centrifugation at 800 rpm for 30 min;
4) 24 hours after the cells were infected by viruses, the culture medium was changed to a 2 mL fresh Polybrene culture medium;
5) 72 hours after the cells were infected by viruses, FACS was performed to detect the percentage of positive cells expressing enhanced green fluorescent protein (EGFP); and
6) groups with the infection rate in a range of 1% to 40% were selected for titer calculation. The calculation formula is:

virus titer=initial cell number×% EGFP×1.5/dilution factor (wherein % EGFP represents the percentage of positive cells expressing EGFP, and the dilution factor is as shown in Table 4).

TABLE 4

| | | | Culture medium |
|---|---|---|---|
| No. | Dilution factor | Virus volume | containing Polybrene |
| 1 | 1 × 10⁻² | 22 μL | 2178 μL |
| 2 | 1 × 10⁻³ | 220 μL of Tube #1 | 1980 μL |
| 3 | 1 × 10⁻⁴ | 220 μL of Tube #2 | 1980 μL |
| 4 | 1 × 10⁻⁵ | 220 μL of Tube #3 | 1980 μL |
| 5 | 1 × 10⁻⁶ | 220 μL of Tube #4 | 1980 μL |

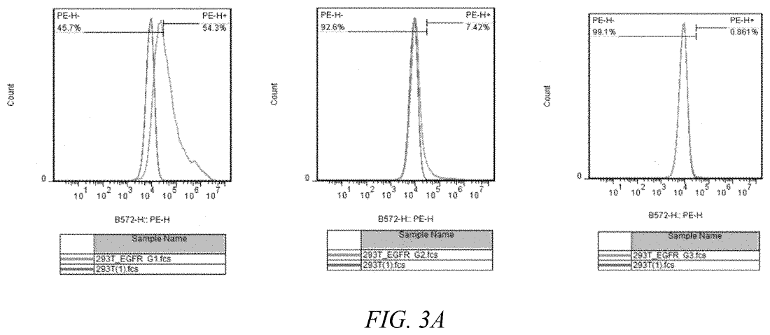

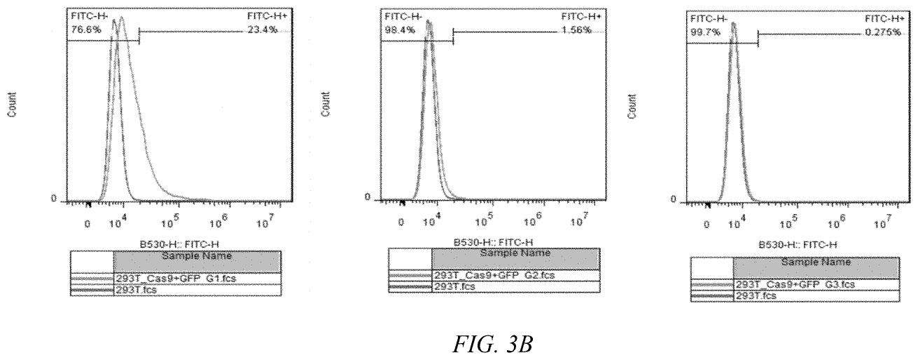

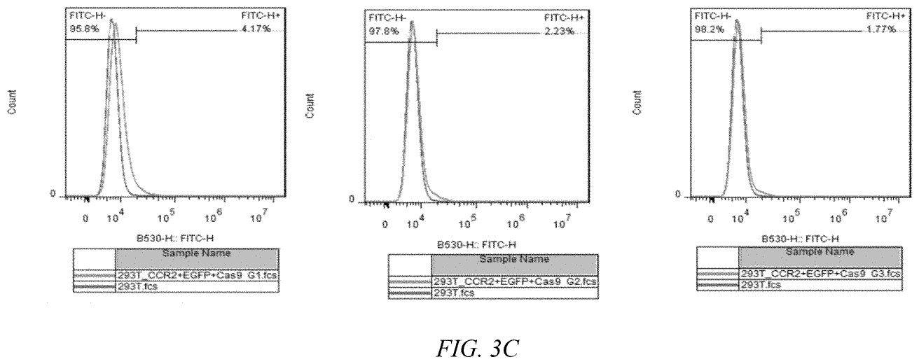

The experimental results are shown in FIGS. 3A to 3C, which indicate that the improved lentiviral expression vector has good repeatability. According to the calculation results of the virus titer as shown in Table 5, from the perspective of the functional titer, if the inserted fragment exceeds 5 kb, although a high physical titer can be obtained, the functional titer will be greatly reduced. Since there is no suitable antibody for Cas9, the functional titer is not detected in this group.

TABLE 5

| | Length | p24 | |
|---|---|---|---|
| | of inserted | physical | FACS |
| | fragments | titer | functional titer |
| Name of | (+ 498 bps | (× 10\hat{ }6 | (× 10\hat{ }6 |
| vectors | flag&P2A&blast) | IFU/mL) | TU/mL) |
| EGFR | 3773 bps | 343.0155 | 333 |
| Cas9 | 4698 bps | 358.608875 | NA |
| Cas9 + EGFP | 5498 bps | 184.65725 | 10.25 |
| CCR2 + | 6622 bps | 261.170125 | 1.87 |
| EGFP + Cas9 | | | |

The sequence information involved in the present invention is as follows:

CMV enhancer (SEQ ID NO: 1):
GACATTGATTATTGACTAGTTATTAATAGTAATCAATTAC
GGGGTCATTAGTTCATAGCCCATATATGGAGTTCCGCGTT
ACATAACTTACGGTAAATGGCCCGCCTGGCTGACCGCCCA
ACGACCCCCGCCCATTGACGTCAATAATGACGTATGTTCC
CATAGTAACGCCAATAGGGACTTTCCATTGACGTCAATGG
GTGGAGTATTTACGGTAAACTGCCCACTTGGCAGTACATC
AAGTGTATCATATGCCAAGTACGCCCCCTATTGACGTCAA
TGACGGTAAATGGCCCGCCTGGCATTATGCCCAGTACATG
ACCTTATGGGACTTTCCTACTTGGCAGTACATCTACGTAT
TAGTCATCGCTATTACCATG
CMV promoter (SEQ ID NO: 2):
GTGATGCGGTTTTGGCAGTACATCAATGGGCGTGGATAGC
GGTTTGACTCACGGGGATTTCCAAGTCTCCACCCCATTGA
CGTCAATGGGAGTTTGTTTTGGCACCAAAATCAACGGGAC
TTTCCAAAATGTCGTAACAACTCCGCCCCATTGACGCAAA
TGGGCGGTAGGCGTGTACGGTGGGAGGTCTATATAAGCAG
CGC
Truncated 5′ LTR (SEQ ID NO: 3)
GGGTCTCTCTGGTTAGACCAGATCTGAGCCTGGGAGCTCT
CTGGCTAACTAGGGAACCCACTGCTTAAGCCTCAATAAAG
CTTGCCTTGAGTGCTTCAAGTAGTGTGTGCCCGTCTGTTG
TGTGACTCTGGTAACTAGAGATCCCTCAGACCCTTTTAGT
CAGTGTGGAAAATCTCTAGCA
EFS promoter (SEQ ID NO: 4)
TAGGTCTTGAAAGGAGTGGGAATTGGCTCCGGTGCCCGTC
AGTGGGCAGAGCGCACATCGCCCACAGTCCCCGAGAAGTT
GGGGGGAGGGGTCGGCAATTGATCCGGTGCCTAGAGAAGG
TGGCGCGGGGTAAACTGGGAAAGTGATGTCGTGTACTGGC
TCCGCCTTTTTCCCGAGGGTGGGGGAGAACCGTATATAAG
TGCAGTAGTCGCCGTGAACGTTCTTTTTCGCAACGGGTTT
GCCGCCAGAACACAGG
P2A linker sequence (SEQ ID NO: 5):
GGATCCGGCGCAACAAACTTCTCTCTGCTGAAACAAGCCG
GAGATGTCGAAGAGAATCCTGGACCG
Blasticidin resistance gene (SEQ ID NO: 6):
ATGGCCAAGCCTTTGTCTCAAGAAGAATCCACCCTCATTG
AAAGAGCAACGGCTACAATCAACAGCATCCCCATCTCTGA
AGACTACAGCGTCGCCAGCGCAGCTCTCTGTAGCGACGGC
CGCATCTTCACTGGTGTCAATGTATATCATTTTACTGGGG
GACCTTGTGCAGAACTCGTGGTGCTGGGCACTGCTGCTGC
TGCGGCAGCTGGCAACCTGACTTGTATCGTCGCGATCGGA
AATGAGAACAGGGGCATCTTGAGCCCCTGCGGACGGTGCC
GACAGGTGCTTCTCGATCTGCATCCTGGGATCAAAGCCAT
AGTGAAGGACAGTGATGGACAGCCGACGGCAGTTGGGATT
CGTGAATTGCTGCCCTCTGGTTATGTGTGGGAGGGCTAA
3′ LTR sequence with a partially deleted
U3 region (SEQ ID NO: 7):
CTGGAAGGGCTAATTCACTCCCAACGAAGACAAGATATCC
TTGATCTGTGGATCTACCACACACAAGGCTACTTCCCTGA
TTGGCAGAACTACACACCAGGGCCAGGGATCAGATATCCA
CTGACCTTTGGATGGTGCTACAAGCTAGTACCAGTTGAGC
AAGAGAAGGTAGAAGAAGCCAATGAAGGAGAGAACACCCG
CTTGTTACACCCTGTGAGCCTGCATGGGATGGATGACCCG
GAGAGAGAAGTATTAGAGTGGAGGTTTGACAGCCGCCTAG
CATTTCATCACATGGCCCGAGAGCTGCATCCGGACTGTAC
TGGGTCTCTCTGGTTAGACCAGATCTGAGCCTGGGAGCTC
TCTGGCTAACTAGGGAACCCACTGCTTAAGCCTCAATAAA
GCTTGCCTTGAGTGCTTCAAGTAGTGTGTGCCCGTCTGTT
GTGTGACTCTGGTAACTAGAGATCCCTCAGACCCTTTTAG
TCAGTGTGGAAAATCTCTAGCAG
EGFR sequence (SEQ ID NO: 8):
ATGCGACCCTCCGGGACGGCCGGGGCAGCGCTCCTGGCGC
TGCTGGCTGCGCTCTGCCCGGCGAGTCGGGCTCTGGAGGA
AAAGAAAGTTTGCCAAGGCACGAGTAACAAGCTCAGGCAG
TTGGGCACTTTTGAAGATCATTTTCTCAGCCTCCAGAGGA
TGTTCAATAACTGTGAGGTGGTCCTTGGGAATTTGGAATT
ACCTATGTGCAGAGGAATTATGATCTTTCCTTCTTAAAGA
CCATCCAGGAGGTGGCTGGTTATGTCCTCATTGCCCTCAA
CACAGTGGAGCGAATTCCTTTGGAAAACCTGCAGATCATC
AGAGGAAATATGTACTACGAAAATTCCTATGCCTTAGCAG
TCTTATCTAACTATGATGCAAATAAAACCGGACTGAAGGA
GCTGCCCATGAGAAATTTACAGGAAATCCTGCATGGCGCC
GTGCGGTTCAGCAACAACCCTGCCCTGTGCAACGTGGAGA
GCATCCAGTGGCGGGACATAGTCAGCAGTGACTTTCTCAG
CAACATGTTGATGGACTTCCAGAACCACCTGGGCAGCTGC
CAAAAGTGTGATCCAAGCTGTCCCAATGGGAGCTGCTGGG
GTGCAGGAGAGGAGAACTGCCAGAAACTGACCAAAATCAT
CTGTGCCCAGCAGTGCTCCGGGCGCTGCCGTGGCAAGTCC
CCCAGTGACTGCTGCCACAACCAGTGTGCTGCAGGCTGCA
CAGGCCCCCGGGAGAGCGACTGCCTGGTCTGCCGCAAATT
CCGAGACGAAGCCACGTGCAAGGACACCTGCCCCCCACTC
ATGCTCTACAACCCCACCACGTACCAGATGGATGTGAACC
CCGAGGGCAAATACAGCTTTGGTGCCACCTGCGTGAAGAA
GTGTCCCCGTAATTATGTGGTGACAGATCACGGCTCGTGC
GTCCGAGCCTGTGGGGCCGACAGCTATGAGATGGAGGAAG
ACGGCGTCCGCAAGTGTAAGAAGTGCGAAGGGCCTTGCCG
CAAAGTGTGTAACGGAATAGGTATTGGTGAATTTAAAGAC
TCACTCTCCATAAATGCTACGAATATTAAACACTTCAAAA
ACTGCACCTCCATCAGTGGCGATCTCCACATCCTGCCGGT
GGCATTTAGGGGTGACTCCTTCACACATACTCCTCCTCTG
GATCCACAGGAACTGGATATTCTGAAAACCGTAAAGGAAA
TCACAGGGTTTTTGCTGATTCAGGCTTGGCCTGAAAACAG
GACGGACCTCCATGCCTTTGAGAACCTAGAAATCATACGC
GGCAGGACCAAGCAACATGGTCAGTTTTCTCTTGCAGTCG
TCAGCCTGAACATAACATCCTTGGGATTACGCTCCCTCAA
GGAGATAAGTGATGGAGATGTGATAATTTCAGGAAACAAA
AATTTGTGCTATGCAAATACAATAAACTGAAAAAACTGTT
TGGGACCTCCGGTCAGAAAACCAAAATTATAAGCAACAGA
GGTGAAAACAGCTGCAAGGCCACAGGCCAGGTCTGCCATG
CCTTGTGCTCCCCCGAGGGCTGCTGGGGCCGGGAGCCCAG
GGACTGCGTCTCTTGCCGGAATGTCAGCCGAGGCAGGGAA
TGCGTGGACAAGTGGAACCTTCTGGAGGGTGAGCCAAGGG
AGTTTGTGGAGAACTCTGAGTGCATACAGTGCCACCCAGA
GTGCCTGCCTCAGGCCATGAACATCACCTGCACAGGACGG
GGACCAGACAACTGTATCCAGTGTGCCCACTACATTGACG
GCCCCCACTGCGTCAAGACCTGCCCGGCAGGAGTCATGGG
AGAAAACAACACCCTGGTCTGGAAGTACGCAGACGCCGGC
CATGTGTGCCACCTGTGCCATCCAAACTGCACCTACGGAT
GCACTGGGCCAGGTCTTGAAGGCTGTCCAACGAATGGGCC
TAAGATCCCGTCCATCGCCACTGGGATGGTGGGGGCCCTC
CTCTTGCTGCTGGTGGTGGCCCTGGGGATCGGCCTCTTCA
TGCGAAGGCGCCACATCGTTCGGAAGCGCACGCTGCGGAG
GCTGCTGCAGGAGAGGGAGCTTGTGGAGCCTCTTACACCC
AGTGGAGAAGCTCCCAACCAAGCTCTCTTGAGGATCTTGA
AGGAAACTGAATTCAAAAAGATCAAAGTGCTGGGCTCCGG
TGCGTTCGGCACGGTGTATAAGGGACTCTGGATCCCAGAA
GGTGAGAAAGTTAAAATTCCCGTCGCTATCAAGGAATTAA
GAGAAGCAACATCTCCGAAAGCCAACAAGGAAATCCTCGA
TGAAGCCTACGTGATGGCCAGCGTGGACAACCCCCACGTG
TGCCGCCTGCTGGGCATCTGCCTCACCTCCACCGTGCAGC
TCATCACGCAGCTCATGCCCTTCGGCTGCCTCCTGGACTA
TGTCCGGGAACACAAAGACAATATTGGCTCCCAGTACCTG
CTCAACTGGTGTGTGCAGATCGCAAAGGGCATGAACTACT
TGGAGGACCGTCGCTTGGTGCACCGCGACCTGGCAGCCAG
GAACGTACTGGTGAAAACACCGCAGCATGTCAAGATCACA
GATTTTGGGCTGGCCAAACTGCTGGGTGCGGAAGAGAAAG
AATACCATGCAGAAGGAGGCAAAGTGCCTATCAAGTGGAT
GGCATTGGAATCAATTTTACACAGAATCTATACCCACCAG
AGTGATGTCTGGAGCTACGGGGTGACTGTTTGGGAGTTGA
TGACCTTTCGATCCAAGCCATATGACGGAATCCCTGCCAG
CGAGATCTCCTCCATCCTGGAGAAAGGAGAACGCCTCCCT
CAGCCACCCATATGTACCATCGATGTCTACATGATCATGG
TCAAGTGCTGGATGATAGACGCAGATAGTCGCCCAAAGTT
CCGTGAGTTGATCATCGAATTCTCCAAAATGGCCCGAGAC
CCCCAGCGCTACCTTGTCATTCAGGGGGATGAAAGAATGC
ATTTGCCAAGTCCTACAGACTCCAACTTCTACCGTGCCCT
GATGGATGAAGAAGACATGGACGACGTGGTGGATGCCGAC
GAGTACCTCATCCCACAGCAGGGCTTCTTCAGCAGCCCCT
CCACGTCACGGACTCCCCTCCTGAGCTCTCTGAGTGCAAC
CAGCAACAATTCCACCGTGGCTTGCATTGATAGAAATGGG
CTGCAAAGCTGTCCCATCAAGGAAGACAGCTTCTTGCAGC
GATACAGCTCAGACCCCACAGGCGCCTTGACTGAGGACAG
CATAGACGACACCTTCCTCCCAGTGCCTGAATACATAAAC
CAGTCCGTTCCCAAAAGGCCCGCTGGCTCTGTGCAGAATC
CTGTCTATGACAATGAGCCTCTGAACCCCGCGCCCAGCAG
AGACCCACACTACCAGGACCCCCACAGCACTGCAGTGGGC
AACCCCGAGTATCTCAACACTGTCCAGCCCACCTGTGTCA
ACAGCACATTCGACAGCCCTGCCCACTGGGCCCAGAAAGG
CAGCCACCAAATTAGCCTGGACAACCCTGACTACCAGCAG
GACTTCTTTCCCAAGGAAGCCAAGCCAAATGGCATCTTTA
AGGGCTCCACAGCTGAAATGCAGAATACCTAAGGGTCGCG
CCACAAAGCAGTGAATTTATTGGAGCA
Cas9 sequence (SEQ ID NO: 9):
ATGGACAAGAAGTACAGCATCGGCCTGGACATCGGCACCA
ACTCTGTGGGCTGGGCCGTGATCACCGACGAGTACAAGGT
GCCCAGCAAGAAATTCAAGGTGCTGGGCAACACCGACCGG
CACAGCATCAAGAAGAACCTGATCGGAGCCCTGCTGTTCG
ACAGCGGCGAAACAGCCGAGGCCACCCGGCTGAAGAGAAC
CGCCAGAAGAAGATACACCAGACGGAAGAACCGGATCTGC
TATCTGCAAGAGATCTTCAGCAACGAGATGGCCAAGGTGG
ACGACAGCTTCTTCCACAGACTGGAAGAGTCCTTCCTGGT
GGAAGAGGATAAGAAGCACGAGCGGCACCCCATCTTCGGC
AACATCGTGGACGAGGTGGCCTACCACGAGAAGTACCCCA
CCATCTACCACCTGAGAAAGAAACTGGTGGACAGCACCGA
CAAGGCCGACCTGCGGCTGATCTATCTGGCCCTGGCCCAC
ATGATCAAGTTCCGGGGCCACTTCCTGATCGAGGGCGACC
TGAACCCCGACAACAGCGACGTGGACAAGCTGTTCATCCA
GCTGGTGCAGACCTACAACCAGCTGTTCGAGGAAAACCCC
ATCAACGCCAGCGGCGTGGACGCCAAGGCCATCCTGTCTG
CCAGACTGAGCAAGAGCAGACGGCTGGAAAATCTGATCGC
CCAGCTGCCCGGCGAGAAGAAGAATGGCCTGTTCGGAAAC
CTGATTGCCCTGAGCCTGGGCCTGACCCCCAACTTCAAGA
GCAACTTCGACCTGGCCGAGGATGCCAAACTGCAGCTGAG
CAAGGACACCTACGACGACGACCTGGACAACCTGCTGGCC
CAGATCGGCGACCAGTACGCCGACCTGTTTCTGGCCGCCA
AGAACCTGTCCGACGCCATCCTGCTGAGCGACATCCTGAG
AGTGAACACCGAGATCACCAAGGCCCCCCTGAGCGCCTCT
ATGATCAAGAGATACGACGAGCACCACCAGGACCTGACCC
TGCTGAAAGCTCTCGTGCGGCAGCAGCTGCCTGAGAAGTA
CAAAGAGATTTTCTTGGACCAGAGCAAGAACGGCTACGCC
GGCTACATTGACGGCGGAGCCAGCCAGGAAGAGTTCTACA
AGTTCATCAAGCCCATCCTGGAAAAGATGGACGGCACCGA
GGAACTGCTCGTGAAGCTGAACAGAGAGGACCTGCTGCGG
AAGCAGCGGACCTTCGACAACGGCAGCATCCCCCACCAGA
TCCACCTGGGAGAGCTGCACGCCATTCTGCGGCGGCAGGA
AGATTTTTACCCATTCCTGAAGGACAACCGGGAAAAGATC
GAGAAGATCCTGACCTTCCGCATCCCCTACTAGGTGGGCC
CTCTGGCCAGGGGAAACAGCAGATTCGCCTGGATGACCAG
AAAGAGCGAGGAAACCATCACCCCCTGGAACTTCGAGGAA
GTGGTGGACAAGGGCGCTTCCGCCCAGAGCTTCATCGAGC
GGATGACCAACTTCGATAAGAACCTGCCCAACGAGAAGGT
GCTGCCCAAGCACAGCCTGCTGTACGAGTACTTCACCGTG
TATAACGAGCTGACCAAAGTGAAATACGTGACCGAGGGAA
TGAGAAAGCCCGCCTTCCTGAGCGGCGAGCAGAAAAAGGC
CATCGTGGACCTGCTGTTCAAGACCAACCGGAAAGTGACC
GTGAAGCAGCTGAAAGAGGACTACTTCAAGAAAATCGAGT
GCTTCGACTCCGTGGAAATCTCCGGCGTGGAAGATCGGTT
CAACGCCTCCCTGGGCACATACCACGATCTGCTGAAAATT
ATCAAGGACAAGGACTTCCTGGACAATGAGGAAAACGAGG
ACATTCTGGAAGATATCGTGCTGACCCTGACACTGTTTGA
GGACAGAGAGATGATCGAGGAACGGCTGAAAACCTATGCC
CACCTGTTCGACGACAAAGTGATGAAGCAGCTGAAGCGGC
GGAGATACACCGGCTGGGGCAGGCTGAGCCGGAAGCTGAT
CAACGGCATCCGGGACAAGCAGTCCGGCAAGACAATCCTG
GATTTCCTGAAGTCCGACGGCTTCGCCAACAGAAACTTCA
TGCAGCTGATCCACGACGACAGCCTGACCTTTAAAGAGGA
CATCCAGAAAGCCCAGGTGTCCGGCCAGGGCGATAGCCTG
CACGAGCACATTGCCAATCTGGCCGGCAGCCCCGCCATTA
AGAAGGGCATCCTGCAGACAGTGAAGGTGGTGGACGAGCT
CGTGAAAGTGATGGGCCGGCACAAGCCCGAGAACATCGTG
ATCGAAATGGCCAGAGAGAACCAGACCACCCAGAAGGGAC
AGAAGAACAGCCGCGAGAGAATGAAGCGGATCGAAGAGGG
CATCAAAGAGCTGGGCAGCCAGATCCTGAAAGAACACCCC
GTGGAAAACACCCAGCTGCAGAACGAGAAGCTGTACCTGT
ACTACCTGCAGAATGGGCGGGATATGTACGTGGACCAGGA
ACTGGACATCAACCGGCTGTCCGACTACGATGTGGACCAT
ATCGTGCCTCAGAGCTTTCTGAAGGACGACTCCATCGACA
ACAAGGTGCTGACCAGAAGCGACAAGAACCGGGGCAAGAG
CGACAACGTGCCCTCCGAAGAGGTCGTGAAGAAGATGAAG
AACTACTGGCGGCAGCTGCTGAACGCCAAGCTGATTACCC
AGAGAAAGTTCGACAATCTGACCAAGGCGGAGAGAGGCGG
CCTGAGCAACTGGATAAGGGCGGCTTCATCAAGAGACAGC
TGGTGGAAACCCGGCAGATCACAAAGCACGTGGCACAGAT
CCTGGACTCCCGGATGAACACTAAGTACGACGAGAATGAC
AAGCTGATCCGGGAAGTGAAAGTGATCACCCTGAAGTCCA
AGCTGGTGTCCGATTTCCGGAAGGATTTCCAGTTTTACAA
AGTGCGCGAGATCAACAACTACCACCACGCCCACGACGCC
TACCTGAACGCCGTCGTGGGAACCGCCCTGATCAAAAAGT
ACCCTAAGCTGGAAAGCGAGTTCGTGTACGGCGACTACAA
GGTGTACGACGTGCGGAAGATGATCGCCAAGAGCGAGCAG
GAAATCGGCAAGGCTACCGCCAAGTACTTCTTCTACAGCA
ACATCATGAACTTTTTCAAGACCGAGATTACCCTGGCCAA
CGGCGAGATCCGGAAGCGGCCTCTGATCGAGACAAACGGC
GAAACCGGGGAGATCGTGTGGGATAAGGGCCGGGATTTTG
CCACCGTGCGGAAAGTGCTGAGCATGCCCCAAGTGAATAT
CGTGAAAAAGACCGAGGTGCAGACAGGCGGCTTCAGCAAA
GAGTCTATCCTGCCCAAGAGGAACAGCGATAAGCTGATCG
CCAGAAAGAAGGACTGGGACCCTAAGAAGTACGGCGGCTT
CGACAGCCCCACCGTGGCCTATTCTGTGCTGGTGGTGGCC
AAAGTGGAAAAGGGCAAGTCCAAGAAACTGAAGAGTGTGA
AAGAGCTACTGGGGATCACCATCATGGAAAGAAGCAGCTT
CGAGAAGAATCCCATCGACTTTCTGGAAGCCAAGGGCTAC
AAAGAAGTGAAAAAGGACCTGATCATCAAGCTGCCTAAGT
ACTCCCTGTTCGAGCTGGAAAACGGCCGGAAGAGAATGCT
GGCCTCTGCCGGCGAACTGCAGAAGGGAAACGAACTGGCC
GTGCCCTCCAAATATGTGAACTTCCTGTACCTGGCCAGCC
ACTATGAGAAGCTGAAGGGCTCGCCCGAGGATAATGAGCA
GAAACAGCTGTTTGTGGAACAGCACAAGGACTACCTGGAC
GAGATCATCGAGCAGATCAGCGAGTTCTCCAAGAGAGTGA
TCCTGGCCGACGCTAATCTGGACAAAGTGCTGTCCGCCTA
CAACAAGCACCGGGATAAGCCCATCAGAGAGCAGGCCGAG
AATATCATCCACCTGTTTACCCTGACCAATCTGGGAGCCC
CTGCCGCCTTCAAGTACTTTGACACCACCATCGACCGGAA
GAGGTACACCAGCACCAAAGAGGTGCTGGACGCCACCCTG
ATCCACCAGAGCATCACCGGCCTGTACGAGACACGGATCG
ACCTGTCTCAGCTGGGAGGCGACAAGCGACCTGCCGCCAC
AAAGAAGGCTGGACAGGCTAAGAAGAAGAAA
CCR2 sequence (SEQ ID NO: 10):
ATGCTGTCCACATCTCGTTCTCGGTTTATCAGAAATACCA
ACGAGAGCGGTGAAGAAGTCACCACCTTTTTTGATTATGA
TTACGGTGCTCCCTGTCATAAATTTGACGTGAAGCAAATT
GGGGCCCAACTCCTGCCTCCGCTCTACTCGCTGGTGTTCA
TGTTTGGTTTTGTGGGCAACATGCTGGTCGTCCTCATCTT
AATAAACTGCAAAAAGCTGAAGTGCTTGACTGACATTTAC
CTGCTCAACCTGGCCATCTCTGATCTGCTTTTTCTTATTA
CTCTCCCATTGTGGGCTCACTCTGCTGCAAATGAGTGGGT
CTTTGGGAATGCAATGTGCAAATTATTCACAGGGCTGTAT
CACATCGGTTATTTTGGCGGAATCTTCTTCATCATCCTCC
TGACAATCGATAGATACCTGGCTATTGTCCATGCTGTGTT
TGCTTTAAAAGCCAGGACGGTCACCTTTGGGGTGGTGACA
AGTGTGATCACCTGGTTGGTGGCTGTGTTTCCTTCTGTCC
GAGGAATCATGTTTACTAAATGCCAGAAAGAAGATTCTGT
TTATGTCTGTGGCCCTTATTTTCCACGAGGATGGAATAAT
TTCCACACAATAATGAGGAACATTTTGGGGCTGGTCCTGC
CGCTGCTCATCATGGTCATCTGCTACTCGGGAATCCTGAA
AACCCTGCTTCGGTGTCGAAACGAGAAGAAGAGGCATAGG
GCAGTGAGAGTCATCTTCACCATCATGATTGTTTACTTTC
TCTTCTGGACTCCCTATAATATTGTCATTCTCCTGAACAC
CTTCCAGGAATTCTTCGGCCTGAGTAACTGTGAAAGCACC
AGTCAACTGGACCAAGCCACGCAGGTGACAGAGACTCTTG
GGATGACTCACTGCTGCATCAATCCCATCATCTATGCCTT
CGTTGGGGAGAAGTTCAGAAGCCTTTTTCACATAGCTCTT
GGCTGTAGGATTGCCCCACTCCAAAAACCAGTGTGTGGAG
GTCCAGGAGTGAGACCAGGAAAGAATGGTGAAAGTGACTA
CACAAGGACTCCTCGATGGTCGTGGAAAAGGAAAGTCAAT
TGGCAGAGCCCCTGAAGCCAGTCTTCAGGACAAAGAAGGA
GCC
Flag sequence (SEQ ID NO: 11):
GATTACAAAGACGATGACGATAAGGATTACAAAGACGATG
ACGATAAGGATTACAAAGACGATGACGATAAG
EGFP sequence (SEQ ID NO: 12):
GCCACCATGGTGAGCAAGGGCGAGGAGCTGTTCACCGGGG
TGGTGCCCATCCTGGTCGAGCTGGACGGCGACGTAAACGG
CCACAAGTTCAGCGTGTCCGGCGAGGGCGAGGGCGATGCC
ACCTACGGCAAGCTGACCCTGAAGTTCATCTGCACCACCG
GCAAGCTGCCCGTGCCCTGGCCCACCCTCGTGGACCACCC
TGACCTACGGCGTGCAGTGCTTCAGCCGCTACCCCGACCA
CATGAAGCAGCACGACTTCTTCAAGTCCGCCATGCCCGAA
GGCTACGTCCAGGAGCGCACCATCTTCTTCAAGGACGACG
GCAACTACAAGACCCGCGCCGAGGTGAAGTTCGAGGGCGA
CACCCTGGTGAACCGCATCGAGCTGAAGGGCATCGACTTC
AAGGAGGACGGCAACATCCTGGGGCACAAGCTGGAGTACA
ACTACAACAGCCACAACGTCTATATCATGGCCGACAAGCA
GAAGAACGGCATCAAGGTGAACTTCAAGATCCGCCACAAC
ATCGAGGACGGCAGCGTGCAGCTCGCCGACCACTACCAGC
AGAACACCCCCATCGGCGACGGCCCCGTGCTGCTGCCCGA
CAACCACTACCTGAGCACCCAGTCCGCCCTGAGCAAAGAC
CCCAACGAGAAGCGCGATCACATGGTGCTGCTGGAGTTGG
TGACCGCCGCCGGGATCACTCTCGGGATGGACGAGCTGT

1: A lentiviral expression vector improved on the basis of PLVX-Puro, wherein the vector comprises at least one, at least two or three of the following improved element sequences:
(1) a CMV promoter sequence upstream of 5′ LTR,
(2) an EFS promoter sequence as a target gene promoter, and
(3) a P2A linker sequence downstream of a multiple cloning site.

2: The lentiviral expression vector according to claim 1, further comprising a CMV enhancer sequence upstream of the CMV promoter sequence upstream of the 5′ LTR.

3: The lentiviral expression vector according to claim 1, wherein the 5′ LTR is a truncated PLVX-Puro 5′ LTR sequence.

4: The lentiviral expression vector according to claim 1, further comprising a 3′ LTR sequence with a partially deleted U3 region.

5: The lentiviral expression vector according to claim 1, wherein the CMV promoter sequence upstream of the 5′ LTR comprises a nucleotide sequence shown in SEQ ID NO: 2 or a nucleotide sequence that has at least 80% identity to the nucleotide sequence shown in SEQ ID NO: 2 and has a promoter activity.

6: The lentiviral expression vector according to claim 1, wherein the EFS promoter sequence comprises a nucleotide sequence shown in SEQ ID NO: 4 or a nucleotide sequence that has at least 80% identity to the nucleotide sequence shown in SEQ ID NO: 4 and has a promoter activity.

7: The lentiviral expression vector according to claim 1, wherein the P2A linker sequence comprises a nucleotide sequence shown in SEQ ID NO: 5 or a nucleotide sequence that has at least 80% identity to the nucleotide sequence shown in SEQ ID NO: 5 and has a ligation function.

8: The lentiviral expression vector according to claim 2, wherein the CMV enhancer sequence comprises a nucleotide sequence shown in SEQ ID NO: 1 or a nucleotide sequence that has at least 80% identity to the nucleotide sequence shown in SEQ ID NO: 1 and has an enhancer activity.

9: The lentiviral expression vector according to claim 3, wherein the truncated 5′ LTR sequence comprises a nucleotide sequence shown in SEQ ID NO: 3 or a nucleotide sequence that has at least 80% identity to the nucleotide sequence shown in SEQ ID NO: 3 and can function as the 5′ LTR.

10: The lentiviral expression vector according to claim 4, wherein the 3′ LTR sequence with the partially deleted U3 region comprises a nucleotide sequence shown in SEQ ID NO: 7 or a nucleotide sequence that has at least 80% identity to the nucleotide sequence shown in SEQ ID NO: 7 and can function as the 3′ LTR.

11: The lentiviral expression vector according to claim 1, wherein the vector further comprises a Blasticidin resistance gene sequence in place of a Puro resistance gene sequence.

12: The lentiviral expression vector according to claim 11, wherein the Blasticidin resistance gene sequence comprises a nucleotide sequence shown in SEQ ID NO: 6 or a nucleotide sequence that has at least 80% identity to the nucleotide sequence shown in SEQ ID NO: 6 and has resistance.

13: A method of improving a lentiviral expression vector on the basis of PLVX-Puro, the method comprising:
(1) adding a CMV promoter sequence to the upstream of 5′ LTR,
(2) replacing a CMV promoter of a target gene with an EFS promoter sequence, and
(3) deleting a PGK promoter downstream of a multiple cloning site and introducing a P2A linker sequence.

14: The method according to claim 13, further comprising one or more of the following improving steps:
(1) adding a CMV enhancer sequence to the upstream of the 5′ LTR,
(2) replacing the original 5′ LTR with a truncated 5′ LTR sequence, and
(3) replacing original 3′ LTR with a 3′ LTR sequence with a partially deleted U3 region.

15: The method according to claim 13, further comprising replacing a Puro resistance gene with a Blasticidin resistance gene sequence.

16: The method according to claim 13, wherein the CMV promoter sequence added comprises a nucleotide sequence that has at least 80% identity to a nucleotide sequence shown in SEQ ID NO: 2 and has a promoter activity; the EFS promoter sequence comprises a nucleotide sequence that has at least 80% identity to a nucleotide sequence shown in SEQ ID NO: 4 and has a promoter activity; and/or the P2A linker sequence comprises a nucleotide sequence that has at least 80% identity to a nucleotide sequence shown in SEQ ID NO: 5 and has a ligation function.

17: The method according to claim 14, wherein the CMV enhancer sequence added comprises a nucleotide sequence that has at least 80% identity to a nucleotide sequence shown in SEQ ID NO: 1 and has an enhancer function; the truncated 5′ LTR sequence comprises a nucleotide sequence that has at least 80% identity to a nucleotide sequence shown in SEQ ID NO: 3 and can function as the 5′ LTR; and/or the 3′ LTR sequence with the partially deleted U3 region comprises a nucleotide sequence that has at least 80% identity to a nucleotide sequence shown in SEQ ID NO: 7 and can function as the 3′ LTR.

18. (canceled)

19: A eukaryotic cell transfected with the lentiviral expression vector according to claim 1.

20. (canceled)

21: Use of the lentiviral expression vector according to claim 1, in the preparation of lentiviral particles.

22. (canceled)

23: Use of the lentiviral expression vector according to claim 1, for preparing a gene or cell therapy drug.
